(19) United States
(12) Patent Application Publication
(10) Pub. No.: US 2026/0167676 A1
(43) Pub. Date: Jun. 18, 2026

(54) COMPOSITIONS COMPRISING HIV-1 MEMBRANE PROXIMAL EXTERNAL REGION (MPER) PEPTIDES AND NUCLEIC ACIDS ENCODING MPER PEPTIDES

(71) Applicants: Duke University, Durham, NC (US); Triad National Security, LLC,, Los Alamos, NM (US)

(72) Inventors: Barton F. HAYNES, Durham, NC (US); Kevin O. SAUNDERS, Durham, NC (US); Zekun MU, Durham, NC (US); Rory HENDERSON, Durham, NC (US); Bette T. Korber, Los Alamos, NM (US)

(21) Appl. No.: 19/123,730

(22) PCT Filed: Oct. 24, 2023
(86) PCT No.: PCT/US2023/077686

§ 371 Date: Apr. 23, 2025

Related U.S. Application Data
(60) Provisional application No. 63/380,730, filed on Oct. 24, 2022.

(51) Int. Cl. C07K 14/005; A61K 38/00

(52) U.S. Cl. CPC C07K 14/005; A61K 38/00; C07K 2317/76

(57) ABSTRACT

In certain aspects the invention provides HIV-1 immunogens, including one or more HIV-1 MPER peptides or HIV-1 envelope protein sequence comprising a MPER peptide for antibody induction.

This International Patent Application claims the benefit of and priority to U.S. Application No. 63/380,730, filed Oct. 24, 2022, entitled “Compositions Comprising HIV-1 Membrane Proximal External Region (MPER) Peptides and Nucleic Acids Encoding MPER peptides,” the content of which is hereby incorporated by reference in its entirety.

This invention was made with government support under Center for HIV/AIDS Vaccine Immunology-Immunogen Design grant UM1-AI144371 from the NIH, NIAID, Division of AIDS. The government has certain rights in the invention.

### TECHNICAL FIELD

The present invention relates in general, to a composition suitable for use in inducing anti-HIV-1 antibodies, and, in particular, to immunogenic compositions comprising nucleic acids, encoding HIV-1 membrane proximal external region (MPER) peptides to induce cross-reactive neutralizing antibodies and increase their breadth of coverage. The invention also relates to methods of inducing such broadly neutralizing anti-HIV-1 antibodies using such compositions.

### BACKGROUND

One of the major challenges to HIV-1 vaccine development has been the inability of immunogens to induce broadly neutralizing antibodies (BnAb). BnAbs are generated during HIV-1 infection. However, most of the BnAbs generated neutralize only the autologous viruses or closely related strains (Moog et al, J. Virol. 71:3734-3741 (1997), Gray et al, J. Virol. 81:6187-6196 (2007)). HIV envelope (Env) constantly mutates to escape from existing BnAb response (Albert et al, Aids 4:107-112 (1990), Wei et al, Nature 422:307-312) (2003)). BnAb responses do evolve over the course of the HIV infection. However, with the mutation capacity of HIV-1 viruses, neutralizing antibody responses always seem to “lag behind” virus evolution (Wei et al, Nature 422:307-312 (2003)), Richman et al, Proc. Natl. Acad. Sci. USA 100:4144-4149 (2003), Geffin et al, Virology 310:207-215 (2003)).

After extensive research, a handful of broadly neutralizing monoclonal antibodies (mAbs) against HIV-1 have been identified (Buchacher et al, AIDS Res. Hum. Retroviruses 10:359-369 (1994), Zwick et al, J. Virol. 75:10892-10895 (2001), Burton et al, Proc. Natl. Acad. Sci. USA 888:10134-10137 (1991)). Two such antibodies, 2F5 and 4E10, target the conserved membrane-proximal external region (MPER) of HIV-1, have a broad spectrum of neutralization (Binley et al, J. Virol. 78:13232-13252 (2004)), and have been shown to neutralize 80% and 100% of newly transmitted viruses (Mehandru et al, J. Virol. 78:14039-14042 (2004)), respectively. When passively administered in combination with several other broadly neutralizing monoclonal antibodies, a cocktail of mAbs composed of 2G12, 2F5 and 4E10 successfully protected the host from virus infection in animal models (Baba et al, Nat. Med. 6:200-206 (2000), Ferrantelli et al, J. Infect. Dis. 189:2167-2173 (2004), Mascola et al, Nat. Med. 6:207-210 (2000), Ruprecht et al, Vaccine 21:3370-3373 (2003)), or delayed virus rebound after cessation of antiretroviral therapy (Trkola et al, Nat. Med. 11:615-622 (2005)).

The potential of using 2F5 and 4E10 to prevent HIV infection is greatly compromised by the fact that HIV infected patients rarely develop these antibodies spontaneously (Dhillon et al, J. Virol. 81:6548-6562 (2007)), and there has been no success in inducing 2F5- and 4E10-like antibodies by vaccination (Kim et al, Vaccine 25:5102-5114 (2006), Coeffier et al, Vaccine 19:684-693 (2000), Joyce et al, J. Biol. Chem. 277:45811-45820 (2002), Ho et al, Vaccine 23:1559-1573 (2005), Zhang et al, Immunobiology 210:639-645 (2005)). Identification of subjects that develop 2F5- or 4E10-like antibodies during natural HIV-1 infection, and developing an understanding of the mechanism of, or hindrance to, these broadly neutralizing antibodies is important for AIDS vaccine design.

The development of a safe and effective MPER immunogens that can induce cross-reactive (broadly) neutralizing Ab (BnAb) is one of the highest priorities of the scientific community working on the HIV-1 epidemic. As many MPERs may be recognized by BnAbs as transmembrane proteins, MPER immunogens in hosts are useful in developing bNAb induction.

### SUMMARY OF THE INVENTION

In certain embodiments, the invention provides compositions and method for induction of immune response, for example cross-reactive (broadly) neutralizing Ab (BnAb) induction.

In certain aspects, the invention provides one or more HIV-1 membrane proximal external region (MPER) peptides. In certain aspects, the invention provides one or more nucleic acids encoding one or more HIV-1 MPER peptides. In certain aspects, the invention provides one or more HIV-1 envelopes proteins comprising a MPER peptide. In certain aspects, the invention provides one or more nucleic acids encoding one or more HIV-1 envelope proteins comprising a MPER peptide.

In certain aspects, the invention provides a method of administering to a patient in need thereof one or more HIV-1 MPER peptides. In certain aspects, the invention provides a method of administering to a patient in need thereof one or more nucleic acids encoding one or more HIV-1 MPER peptides. In certain aspects, the invention provides a method of administering to a patient in need thereof one or more HIV-1 envelopes proteins comprising a MPER peptide. In certain aspects, the invention provides a method of administering to a patient in need thereof one or more nucleic acids encoding one or more HIV-1 envelope proteins comprising a MPER peptide. In certain embodiments, the patient is infected with HIV (e.g., HIV-1).

In certain embodiments, the patient in need thereof is an HIV-uninfected individual. In certain embodiments, the patient in need thereof is an HIV-infected individual. In certain embodiments, the administration to the HIV-infected induces individual broadly neutralizing antibodies. In certain embodiments the broadly neutralizing antibodies of the HIV-infected individual mediates viral (e.g., HIV-1) clearance from blood and tissues.

In certain embodiments, the method provides administering the one or more MPER peptides or the one or more HIV-1 envelopes proteins comprising a MPER peptide sequentially as a prime and/or as a boost. In certain embodiments, the method provides administering the one or more nucleic acids encoding one or more MPER peptides or encoding one or more HIV-1 envelopes proteins comprising a MPER peptide sequentially as a prime and/or as a boost. In certain embodiments, the method provides administering the one or more MPER peptides or the one or more HIV-1 envelopes proteins comprising a MPER peptide non-sequentially. In certain embodiments, the method provides administering the one or more nucleic acids encoding one or more MPER peptides or encoding one or more HIV-1 envelopes proteins comprising a MPER peptide non-sequentially.

In certain embodiments the one or more nucleic acids encoding the one or more MPER peptide or encoding one or more HIV-1 envelopes proteins comprising a MPER peptide are operably linked to a promoter inserted in an expression vector. In some embodiments, the expression vector comprises DNA. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1. Various assays that analyze whether an immunogenic composition induces an immune response, and the type of antibodies induced are known in the art and are also described herein.

In certain aspects the invention provides a nucleic acid sequence encoding any of the polypeptides of the invention, wherein the nucleic acid is operably linked to a promoter. In certain aspects the invention provides a nucleic acid consisting essentially of a nucleic acid sequence encoding any of the polypeptides of the invention, wherein the nucleic acid is operably linked to a promoter. In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain aspects the invention provides an expression vector consisting essentially a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector. In some embodiments, the expression vector comprises DNA.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In certain aspects the invention provides a composition comprising at least one nucleic acid encoding an HIV-1 MPER peptide or encoding at least one HIV-1 envelope protein comprising a MPER peptide of the invention.

In certain embodiments, the compositions and methods employ an HIV-1 MPER as polypeptide instead of a nucleic acid sequence encoding the HIV-1 MPER. In certain embodiments, the compositions and methods employ an HIV-1 MPER as polypeptide, a nucleic acid sequence encoding the HIV-1 MPER, or a combination thereof. In certain embodiments, the polypeptides are recombinantly produced.

In certain embodiments, the compositions and methods employ an HIV-1 MPER as part of a HIV-1 envelope polypeptide. In certain embodiments, the compositions and methods employ as part of a HIV-1 envelope polypeptide, a nucleic acid sequence encoding the HIV-1 MPER as part of a HIV-1 envelope polypeptide, or a combination thereof. In certain embodiments, the HIV-1 envelope polypeptides are recombinantly produced. In certain embodiments, the HIV-1 envelope polypeptide is a full-length gp160. The envelope used in the compositions and methods of the invention can be a gp160, gp150, gp140, gp145 (i.e, with a transmembrane domain), gp120, gp41 or N-terminal deletion variants thereof as described herein, cleavage resistant variants thereof as described herein, or codon optimized sequences thereof. In certain embodiments, the HIV-1 envelope polypeptide comprises a heptad repeat 2 region of HIV gp41 Env.

The polypeptide contemplated by the invention can be a polypeptide comprising any one of the polypeptides described herein. The polypeptide contemplated by the invention can be a polypeptide consisting essentially of any one of the polypeptides described herein. The polypeptide contemplated by the invention can be a polypeptide consisting of any one of the polypeptides described herein. In certain embodiments, the polypeptide is recombinantly produced. In certain embodiments, the polypeptides and nucleic acids of the invention are suitable for use as an immunogen, for example to be administered in a human subject.

In certain embodiments, the envelope is in a liposome. In certain embodiments the envelope comprises a transmembrane domain with a cytoplasmic tail embedded in a liposome.

In certain embodiments, where the nucleic acids are operably linked to a promoter and inserted in a vector, the vectors are any suitable vector. Non-limiting examples include, VSV, replicating rAdenovirus type 4, MVA, Chimp adenovirus vectors, pox vectors, and the like. In some embodiments, the vector comprises DNA. In certain embodiments, the nucleic acids are administered in NanoTaxi block polymer nanospheres. In certain embodiments, the composition and methods comprise an adjuvant. Non-limiting examples include, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339). In certain embodiments, the TLR 9 ligand is oligo CpG. In certain embodiments, the TLR ligand is a TLR 4 ligand. In certain embodiments, the TLR 4 ligand is monophosphorylipid A.

In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi:10.3390/vaccines8030433, published 3 Aug. 2020. In non-limiting embodiments, LNPs used as adjuvants for proteins composition are composed of an ionizable lipid, cholesterol, lipid conjugated with polyethylene glycol, and a helper lipid. Non-limiting embodiment include LNPs without polyethylene glycol.

In certain aspects the invention provides a cell comprising a nucleic acid encoding any one of the MPER peptides of the invention suitable for recombinant expression. In certain aspects the invention provides a cell comprising a nucleic acid encoding any one the HIV-1 envelope proteins comprising a MPER peptide. In certain aspects, the invention provides a clonally derived population of cells encoding any one of the MPER peptides or HIV-1 envelope proteins comprising a MPER peptide of the invention suitable for recombinant expression. In certain aspects, the invention provides a stable pool of cells encoding any one of the MPER peptides or HIV-1 envelope proteins comprising a MPER peptide of the invention suitable for recombinant expression.

In certain aspects, the invention provides a recombinant HIV-1 MPER polypeptide or HIV-1 envelope proteins comprising a MPER peptide listed in FIG. 4. In certain embodiments, the polypeptide is a non-naturally occurring protomer designed to form an envelope trimer. The invention also provides nucleic acids encoding these recombinant polypeptides.

In certain aspects the invention provides an immunogenic composition comprising a nucleic acid encoding a recombinant HIV-1 MPER peptide and a carrier. In certain aspects the invention provides an immunogenic composition comprising a nucleic acid encoding a recombinant HIV-1 envelope protein comprising a MPER peptide and a carrier.

In certain aspects the invention provides nucleic acids encoding HIV-1 MPER peptide for immunization wherein the nucleic acid encodes a soluble, stabilized, or a transmembrane bound MPER peptide.

In certain aspects the invention provides nucleic acids encoding HIV-1 envelope protein comprising a MPER peptide for immunization wherein the nucleic acid encodes a gp145, gp150, or gp160 transmembrane bound HIV-1 envelope protein comprising a MPER peptide.

In certain embodiments, the compositions for use in immunization further comprise an adjuvant.

In certain embodiments, wherein the compositions comprise a nucleic acid, the nucleic acid is operably linked to a promoter, and could be inserted in an expression vector.

In one aspect the invention provides a composition for a prime boost immunization regimen comprising one or more MPER peptides or HIV-1 envelope proteins comprising a MPER peptide from FIG. 4 and/or one or more nucleic acids encoding the one or more MPER peptides or HIV-1 envelope proteins comprising a MPER peptide from FIG. 4, wherein the MPER peptide or HIV-1 envelope proteins comprising a MPER peptide or nucleic acid encoding the MPER peptide or HIV-1 envelope proteins comprising a MPER peptide is a prime or boost immunogen. In one aspect the invention provides a composition for a prime boost immunization regimen comprising one or more MPER peptides or HIV-1 envelope proteins comprising a MPER peptide or one or more nucleic acids encoding one or more MPER peptides or HIV-1 envelope proteins comprising a MPER peptide of the invention.

In certain aspects the invention provides methods of inducing an immune response in a subject comprising administering a composition comprising a polypeptide and/or any suitable form of a nucleic acid(s) encoding an HIV-1 MPER(s) or HIV-1 envelope proteins comprising a MPER peptide in an amount sufficient to induce an immune response.

In certain embodiments, the nucleic acid encodes a soluble, stabilized, or a transmembrane bound MPER.

In certain embodiments, the nucleic acid encodes a HIV-1 envelope protein comprising a MPER peptide for immunization wherein the nucleic acid encodes a gp145, gp150, or gp160 transmembrane bound HIV-1 envelope protein comprising a MPER peptide.

In certain embodiments, the methods comprise administering an adjuvant. In certain embodiments, the methods comprise administering an agent which modulates host immune tolerance. In certain embodiments, the administered polypeptide is multimerized in a liposome or nanoparticle. In certain embodiments, the methods comprise administering one or more additional HIV-1 immunogens to induce a T cell response. Non-limiting examples include gag, nef, pol, etc.

In certain aspects, the invention provides a composition comprising any one of the inventive MPERs or nucleic acid sequences encoding the same.

In certain aspects, the invention provides a method of inducing an immune response in a subject comprising administering an immunogenic composition comprising any one of the stabilized MPER peptides or HIV-1 envelope proteins comprising a MPER peptide of the invention. In certain embodiments, the composition is administered as a prime and/or a boost. In certain embodiments, the composition comprises nanoparticles. In certain embodiments, methods of the invention further comprise administering an adjuvant.

In certain aspects, the invention provides nucleic acids comprising sequences encoding polypeptides or proteins of the invention. In certain embodiments, the nucleic acids are DNAs. In certain aspects, the invention provides expression vectors comprising the nucleic acids of the invention.

In certain aspects the invention provides nucleic acids encoding the inventive polypeptide or protein designs.

In non-limiting embodiments, the invention provides compositions comprising an MPER peptide or HIV-1 envelope proteins comprising a MPER peptide selected from FIG. 4, or any combination thereof. Provided are also methods of using these MPERs or HIV-1 envelope proteins comprising a MPER peptide and/or nucleic acids, and/or compositions comprising administering an amount sufficient to induce immune responses in a subject.

In some embodiments, the invention provides a recombinant HIV-1 MPER peptide or HIV-1 envelope protein sequence according to any one of Strategies 1, 2, 3, or 4 of FIG. 3-1.

In some embodiments, the invention provides a recombinant HIV-1 MPER peptide or HIV-1 envelope protein sequence comprising MPER-Th epitope as shown in FIG. 3-1. In some embodiments, the recombinant HIV-1 MPER peptide or HIV-1 envelope protein sequence comprises MPER, gag α-helix region 1 (GTH1), gag α-helix region 2 (GTH2), pan HLA DR-binding epitope (PADRE), transmembrane domain (TMD), transmembrane domain (TMD) with Y712I mutation or any combination thereof. In some embodiments, the recombinant HIV-1 MPER peptide or HIV-1 envelope protein sequence comprises MPER and GTH1. In some embodiments, the recombinant HIV-1 MPER peptide or HIV-1 envelope protein sequence comprises MPER, GTH1 and PADRE. In some embodiments, the recombinant HIV-1 MPER peptide or HIV-1 envelope protein sequence comprises MPER, GTH1 and GTH2. In some embodiments, the recombinant HIV-1 MPER peptide or HIV-1 envelope protein sequence comprises MPER and GTH2. In some embodiments, the recombinant HIV-1 MPER peptide or HIV-1 envelope protein sequence comprises MPER and TMD. In some embodiments, the recombinant HIV-1 MPER peptide or HIV-1 envelope protein sequence comprises MPER and TMD with Y712I mutation. In some embodiments, the recombinant HIV-1 MPER peptide or HIV-1 envelope protein sequence comprises PADRE, MPER and TMD. In some embodiments, the recombinant HIV-1 MPER peptide or HIV-1 envelope protein sequence comprises PADRE, MPER and TMD with Y712I mutation. In some embodiments, the recombinant HIV-1 MPER peptide or HIV-1 envelope protein sequence comprises GTH2, MPER and TMD. In some embodiments, the recombinant HIV-1 MPER peptide or HIV-1 envelope protein sequence comprises GTH2, MPER and TMD with Y712I mutation. In some embodiments, MPER comprises a MPER.ConB, a MPER.ConC or any one of the MPERs from FIG. 4. The recombinant HIV-1 MPER peptide or HIV-1 envelope protein sequence comprises MPER, GTH1, GTH2, PADRE, TMD, TMD with Y712I mutation or any combination thereof, in any N to C terminal order. In some embodiments, the N to C terminal order of the domains is depicted in FIG. 3-1.

In some embodiments, the invention provides a recombinant HIV-1 MPER peptide or HIV-1 envelope protein sequence comprising MPER-TMD700 as shown in FIG. 3-1. In some embodiments, the recombinant HIV-1 MPER peptide or HIV-1 envelope protein sequence comprises MPER, transmembrane domain 700 (TMD700), TMD700 with Y712I mutation or any combination thereof. In some embodiments, the recombinant HIV-1 MPER peptide or HIV-1 envelope protein sequence comprises MPER and TMD700. In some embodiments, the recombinant HIV-1 MPER peptide or HIV-1 envelope protein sequence comprises MPER and TMD700 with Y712I mutation. In some embodiments, the recombinant HIV-1 MPER peptide or HIV-1 envelope protein sequence comprises a truncated version of MPER and TMD700 with Y712I mutation. In some embodiments, MPER comprises a MPER.ConB, a MPER.ConC or any one of the MPERs from FIG. 4. In some embodiments, the truncated version of MPER is the distal epitope. The recombinant HIV-1 MPER peptide or HIV-1 envelope protein sequence comprises MPER, TMD700, TMD700 with Y712I mutation or any combination thereof, in any N to C terminal order. In some embodiments, the N to C terminal order of the domains is depicted in FIG. 3-1.

In some embodiments, the invention provides a recombinant HIV-1 MPER peptide or HIV-1 envelope protein sequence comprising MPER-TMD-CD as shown in FIG. 3-1. In some embodiments, the recombinant HIV-1 MPER peptide or HIV-1 envelope protein sequence comprises MPER, transmembrane domain (TMD) with Y712I mutation, cytoplasmic domain (CD), gag α-helix region 1 (GTH1), pan HLA DR-binding epitope (PADRE) or any combination thereof. In some embodiments, the recombinant HIV-1 MPER peptide or HIV-1 envelope protein sequence comprises MPER, TMD with Y712I mutation and CD. In some embodiments, the recombinant HIV-1 MPER peptide or HIV-1 envelope protein sequence comprises MPER, TMD with Y712I mutation, CD and GTH1. In some embodiments, the recombinant HIV-1 MPER peptide or HIV-1 envelope protein sequence comprises MPER, TMD with Y712I mutation, CD and PADRE. In some embodiments, MPER comprises a MPER.ConB, a MPER.ConC, or any one of the MPERs from FIG. 4. The recombinant HIV-1 MPER peptide or HIV-1 envelope protein sequence comprises MPER, TMD with Y712I mutation, CD, GTH1, PADRE, or any combination thereof, in any N to C terminal order. In some embodiments, the N to C terminal order of the domains is depicted in FIG. 3-1.

In some embodiments, the invention provides a recombinant HIV-1 MPER peptide or HIV-1 envelope protein sequence comprising a full length gp160 as shown in FIG. 3-1. In some embodiments, the recombinant HIV-1 MPER peptide or HIV-1 envelope protein sequence comprises a full length gp160, transmembrane domain (TMD) with Y712I mutation or cytoplasmic domain (CD), or any combination thereof. In some embodiments, the recombinant HIV-1 MPER peptide or HIV-1 envelope protein sequence comprises a full length gp160, TMD with Y712I mutation, and a CD. In some embodiments, the full length gp160 comprises an MPER sequence comprising MPER.ConB, a MPER.ConC, or any one of the MPERs from FIG. 4. In some embodiments, the full length gp160 further comprises at least one mutation. In some embodiment, the full length gp160 comprises at least one mutation at position L669, M687, I688, V689, G691 or G696. In some embodiments, the mutation is L669S. In some embodiments, the mutation is M687R. In some embodiments, the mutation is I688R. In some embodiments, the mutation is V689R. In some embodiments, the mutation is G691R. In some embodiments, the mutation is G696R. In some embodiments, the N to C terminal order of the domains is depicted in FIG. 3-1.

In some embodiments, the invention provides a nucleic acid encoding the recombinant HIV-1 MPER peptide or HIV-1 envelope protein sequence according to any one of Strategies 1, 2, 3, or 4 of FIG. 3-1.

In some embodiments, the invention provides a nucleic acid encoding the recombinant HIV-1 MPER peptide or HIV-1 envelope protein sequence comprising MPER-Th epitope as shown in FIG. 3-1. In some embodiments, the nucleic acid encoding the recombinant HIV-1 MPER peptide or HIV-1 envelope protein sequence comprises MPER, gag α-helix region 1 (GTH1), gag α-helix region 2 (GTH2), pan HLA DR-binding epitope (PADRE), transmembrane domain (TMD), transmembrane domain (TMD) with Y712I mutation or any combination thereof. In some embodiments, the nucleic acid encoding the recombinant HIV-1 MPER peptide or HIV-1 envelope protein sequence comprises MPER and GTH1. In some embodiments, the nucleic acid encoding the recombinant HIV-1 MPER peptide or HIV-1 envelope protein sequence comprises MPER, GTH1 and PADRE. In some embodiments, the nucleic acid encoding the recombinant HIV-1 MPER peptide or HIV-1 envelope protein sequence comprises MPER, GTH1 and GTH2. In some embodiments, the nucleic acid encoding the recombinant HIV-1 MPER peptide or HIV-1 envelope protein sequence comprises MPER and GTH2. In some embodiments, the nucleic acid encoding the recombinant HIV-1 MPER peptide or HIV-1 envelope protein sequence comprises MPER and TMD. In some embodiments, the nucleic acid encoding the recombinant HIV-1 MPER peptide or HIV-1 envelope protein sequence comprises MPER and TMD with Y712I mutation. In some embodiments, the nucleic acid encoding the recombinant HIV-1 MPER peptide or HIV-1 envelope protein sequence comprises PADRE, MPER and TMD. In some embodiments, the nucleic acid encoding the recombinant HIV-1 MPER peptide or HIV-1 envelope protein sequence comprises PADRE, MPER and TMD with Y712I mutation. In some embodiments, the nucleic acid encoding the recombinant HIV-1 MPER peptide or HIV-1 envelope protein sequence comprises GTH2, MPER and TMD. In some embodiments, the nucleic acid encoding the recombinant HIV-1 MPER peptide or HIV-1 envelope protein sequence comprises GTH2, MPER and TMD with Y712I mutation. In some embodiments, the MPER peptide encoded by the nucleic acid comprises a MPER.ConB, a MPER.ConC or any one of the MPERs from FIG. 4. The nucleic acid encoding the recombinant HIV-1 MPER peptide or HIV-1 envelope protein sequence comprises MPER, GTH1, GTH2, PADRE, TMD, TMD with Y712I mutation or any combination thereof, in any N to C terminal order. In some embodiments, the N to C terminal order of the domains is depicted in FIG. 3-1.

In some embodiments, the invention provides a nucleic acid encoding a recombinant HIV-1 MPER peptide or HIV-1 envelope protein sequence comprising MPER-TMD700 as shown in FIG. 3-1. In some embodiments, the nucleic acid encoding the recombinant HIV-1 MPER peptide or HIV-1 envelope protein sequence comprises MPER, transmembrane domain 700 (TMD700), TMD700 with Y712I mutation or any combination thereof. In some embodiments, the nucleic acid encoding the recombinant HIV-1 MPER peptide or HIV-1 envelope protein sequence comprises MPER and TMD700. In some embodiments, the nucleic acid encoding the recombinant HIV-1 MPER peptide or HIV-1 envelope protein sequence comprises MPER and TMD700 with Y712I mutation. In some embodiments, the nucleic acid encoding the recombinant HIV-1 MPER peptide or HIV-1 envelope protein sequence comprises a truncated version of MPER and TMD700 with Y712I mutation. In some embodiments, the truncated version of MPER is the distal epitope. In some embodiments, the MPER peptide encoded by the nucleic acid comprises a MPER.ConB, a MPER.ConC or any one of the MPERs from FIG. 4. The nucleic acid encoding the recombinant HIV-1 MPER peptide or HIV-1 envelope protein sequence comprises MPER, TMD700, TMD700 with Y712I mutation or any combination thereof, in any N to C terminal order. In some embodiments, the N to C terminal order of the domains is depicted in FIG. 3-1.

In some embodiments, the invention provides a nucleic acid encoding a recombinant HIV-1 MPER peptide or HIV-1 envelope protein sequence comprising MPER-TMD-CD as shown in FIG. 3-1. In some embodiments, the nucleic acid encoding the recombinant HIV-1 MPER peptide or HIV-1 envelope protein sequence comprises MPER, transmembrane domain (TMD) with Y712I mutation, cytoplasmic domain (CD), gag α-helix region 1 (GTH1), pan HLA DR-binding epitope (PADRE) or any combination thereof. In some embodiments, the nucleic acid encoding the recombinant HIV-1 MPER peptide or HIV-1 envelope protein sequence comprises MPER, TMD with Y712I mutation and CD. In some embodiments, the nucleic acid encoding the recombinant HIV-1 MPER peptide or HIV-1 envelope protein sequence comprises MPER, TMD with Y712I mutation, CD and GTH1. In some embodiments, the nucleic acid encoding the recombinant HIV-1 MPER peptide or HIV-1 envelope protein sequence comprises MPER, TMD with Y712I mutation, CD and PADRE. In some embodiments, the MPER peptide encoded by the nucleic acid comprises a MPER.ConB, a MPER.ConC or any one of the MPERs from FIG. 4. The nucleic acid encoding the recombinant HIV-1 MPER peptide or HIV-1 envelope protein sequence comprises MPER, TMD with Y712I mutation, CD, GTH1, PADRE, or any combination thereof, in any N to C terminal order. In some embodiments, the N to C terminal order of the domains is depicted in FIG. 3-1.

In some embodiments, the invention provides a nucleic acid encoding a recombinant HIV-1 MPER peptide or HIV-1 envelope protein sequence comprising a full length gp160 as shown in FIG. 3-1. In some embodiments, the nucleic acid encoding the recombinant HIV-1 MPER peptide or HIV-1 envelope protein sequence comprises a full length gp160, transmembrane domain (TMD) with Y712I mutation or cytoplasmic domain (CD), or any combination thereof. In some embodiments, the nucleic acid encoding the recombinant HIV-1 MPER peptide or HIV-1 envelope protein sequence comprises a full length gp160, TMD with Y712I mutation, and a CD. In some embodiments, the full length gp160 comprises an MPER sequence comprising MPER.ConB, a MPER.ConC, or any one of the MPERs from FIG. 4. In some embodiments, the full length gp160 further comprises at least one mutation. In some embodiment, the full length gp160 comprises at least one mutation at position L669, M687, I688, V689, G691 or G696. In some embodiments, the mutation is L669S. In some embodiments, the mutation is M687R. In some embodiments, the mutation is I688R. In some embodiments, the mutation is V689R. In some embodiments, the mutation is G691R. In some embodiments, the mutation is G696R. In some embodiments, the N to C terminal order of the domains is depicted in FIG. 3-1.

In some embodiments, the MPER peptide or the MPER peptide encoded by the nucleic acid comprises an MPER.ConB (NEQELLELDKWASLWNWFNITNWLWYIK (SEQ ID NO: 1)). In some embodiments, the MPER peptide or the MPER peptide encoded by the nucleic acid comprises an MPER.ConC (NEKDLLALDSWKNLWNWFDITKWLWYIK (SEQ ID NO: 2)). In some embodiments, the MPER peptide or HIV-1 envelope protein comprising a MPER peptide or the MPER peptide or HIV-1 envelope protein comprising a MPER peptide encoded by the nucleic acid comprises a TMD region. In some embodiments, the MPER peptide or HIV-1 envelope protein comprising a MPER peptide or the MPER peptide or HIV-1 envelope protein comprising a MPER peptide encoded by the nucleic acid comprises a TMD700 region. In some embodiments, the MPER peptide or HIV-1 envelope protein comprising a MPER peptide or the MPER peptide or HIV-1 envelope protein comprising a MPER peptide encoded by the nucleic acid comprises a CD region. In some embodiments, the MPER peptide or the MPER peptide encoded by the nucleic acid comprises a GTH1 region. In some embodiments, the MPER peptide or the MPER peptide encoded by the nucleic acid comprises a GTH2 region. In some embodiments, the MPER peptide or the MPER peptide encoded by the nucleic acid comprises a PADRE region. In some embodiments, the MPER peptide or HIV-1 envelope protein comprising a MPER peptide or the MPER peptide or HIV-1 envelope protein comprising a MPER peptide encoded by the nucleic acid comprises a MPER.ConB or a MPER.ConC and one or more of the TMD, TMD700, CD, GTH1, GTH2, and/or PADRE regions. See FIGS. 3-1 to 3-10.

In some embodiments, the invention provides an immunogenic composition comprising a nucleic acid encoding the HIV-1 MPER peptide or HIV-1 envelope protein comprising a MPER peptide and a carrier. In some embodiments, the compositions comprise at least two different immunogens targeting different UCAs. In non-limiting embodiments, the immunogens are from FIG. 4.

In some embodiments the immunogenic composition further comprises an adjuvant.

In some aspects, the invention provides a method of inducing an immune response in a subject comprising administering a composition comprising any suitable form of a nucleic acid(s) encoding one or more MPER peptide or HIV-1 envelope proteins comprising a MPER peptide selected from FIG. 4 or any combination thereof in an amount sufficient to induce an immune response.

In some embodiments, the composition administered comprises a nucleic acid encoding a soluble, stabilized, or a transmembrane bound MPER peptide.

In some embodiments, the composition administered comprises a nucleic acid encoding a HIV-1 envelope protein comprising a MPER peptide wherein the nucleic acid encodes a gp145, gp150, or gp160 transmembrane bound HIV-1 envelope protein comprising a MPER peptide.

In some embodiments, the composition administered comprises a polypeptide, wherein the polypeptide is a soluble, stabilized, or a transmembrane bound MPER peptide.

In some embodiments, the composition administered comprises a polypeptide encoding a HIV-1 envelope protein comprising a MPER peptide wherein the envelope is a gp145, gp150, or gp160 transmembrane bound HIV-1 envelope protein comprising a MPER peptide.

In some embodiments, the composition administered further comprises an adjuvant.

In some embodiments, the method further comprises administering an agent which modulates host immune tolerance. In some embodiment, the polypeptide administered is multimerized in a liposome or nanoparticle.

In some embodiments, the method further comprising administering one or more additional HIV-1 immunogens to induce a T cell response.

In some aspects, the invention provides a composition comprises a nanoparticle and a carrier, wherein the nanoparticle comprises an MPER peptide or HIV-1 envelope protein comprising a MPER peptide, wherein the MPER peptide or HIV-1 envelope protein comprising a MPER peptide is selected from FIG. 4 or any combination thereof. In some embodiments, the compositions comprises two, three, four or more different immunogens. In some embodiments the immunogens target different UCAs.

In some aspects, the invention provides a composition comprising a nanoparticle and a carrier, wherein the nanoparticle comprises a nucleic acid encoding one or more MPER peptides or HIV-1 envelope proteins comprising a MPER peptide from FIG. 4.

In some embodiments, the nanoparticle of the composition is a ferritin self-assembling nanoparticle.

In some aspects, the invention provides a method of inducing an immune response in a subject comprising administering an immunogenic composition comprising any one of the MPER peptides or HIV-1 envelope proteins comprising a MPER peptide, nucleic acids encoding an MPER peptide or HIV-1 envelope proteins comprising a MPER peptide, or compositions described herein. In some embodiments the methods comprise administering two, three, four or more different immunogens. In some embodiments, the different immunogens target different UCAs. In non-limiting embodiments the different immunogens are selected from FIG. 4.

In some embodiments, the composition is administered as a single prime or as repetitive immunization prime. In preferred embodiments, the repetitive immunization is administered 3 or 4 times.

In some embodiments, the composition is administered as a single boost or as a repetitive series of boosts. In preferred embodiments, the repetitive series of boosts is administered 3 or 4 times.

In some embodiments, the composition is a first composition administered as a prime. In some embodiments, the composition is a second composition administered as one or more boosts. In some embodiments, the method comprises administering the first composition as a prime and administering the second composition as one or more boosts. In preferred embodiments, the first composition and the second composition are different.

In some aspects, the invention provides a nucleic acid encoding any of the MPER peptides or HIV-1 envelope proteins comprising a MPER peptide described herein. In some embodiments, the invention provides a composition comprising the nucleic acid and a carrier.

In some embodiments, the invention provides a method of inducing an immune response in a subject comprising administering an immunogenic composition comprising the nucleic acid encoding any of the MPER peptides or HIV-1 envelope proteins comprising a MPER peptide described herein. In some embodiments, the immunogenic composition further comprises a carrier.

In certain aspects, the invention provides an immunogenic composition or composition, wherein the composition comprises at least two different HIV-1 MPER polypeptides or HIV-1 envelope proteins comprising a MPER peptide or nucleic acids encoding a HIV-1 MPER polypeptide or HIV-1 envelope protein comprising a MPER peptide, or a combination thereof.

In certain aspects, the invention provides an immunogenic composition comprising a first immunogen and a second immunogen, wherein the first immunogen is a HIV-1 MPER peptide or HIV-1 envelope protein comprising a MPER peptide from FIG. 4 or a nucleic acid encoding said HIV-1 MPER peptide or HIV-1 envelope protein comprising a MPER peptide, and wherein the second immunogen is a different HIV-1 MPER peptide or HIV-1 envelope protein comprising a MPER peptide from FIG. 4 or a nucleic acid encoding said different HIV-1 MPER peptide or HIV-1 envelope protein comprising a MPER peptide. In certain aspects, the invention provides a method of inducing an immune response in a subject comprising administering the immunogenic composition in an amount sufficient to induce an immune response. In certain embodiments, the method further comprising administering an agent which modulates host immune tolerance.

In certain embodiments, at least one of the first immunogen and the second immunogen is a HIV-1 MPER peptide. In certain embodiments, at least one of the first immunogen and the second immunogen is a HIV-1 envelope protein comprising a MPER peptide from FIG. 4. In certain embodiments, at least one of the first immunogen and the second immunogen is a nucleic acid. In certain embodiments, the first immunogen and the second immunogen are a nucleic acid. In certain embodiments, the immunogenic composition further comprises one or more additional immunogens, wherein the one or more additional immunogens is different to the first and second immunogens.

In certain embodiments, the HIV-1 MPERs are in the form of a HIV-1 MPER peptides or HIV-1 envelope protein comprising a MPER peptide or nucleic acid, or a combination thereof. In certain embodiments, the composition comprises a carrier. In certain embodiments, the composition further comprises an adjuvant.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIG. 1 discloses the developmental pathway of human B cells. Human B cells arise from committed progenitor cells that proliferate following expression of functional immunoglobulin heavy- (H-) chain polypeptides that associate with surrogate light chains (SLC). In pre-B I cells. H-chain and SLC pairs associate with Igα/Igβ heterodimers to form pre-B cell receptors (pre-BCR) and initiate cell proliferation. When these proliferating cells exit the cell cycle as pre-B II cells, increased RAG1/2 expression drives light- (L-) chain rearrangements and the assembly of mature BCR capable of binding antigen. Most newly generated immature B cells are autoreactive and consequently lost or inactivated at the first tolerance checkpoint; the remainder mature as transitional 1 (T1) and T2 B cells characterized by changes in membrane IgM (mIgM) density, increased mIgD expression, and the loss/diminution of CD10 and CD38. Newly formed T2 B cells are subject to a second round to immune tolerization before entering the mature B cell pools. Mature B cells activated by antigens and TFH characteristically down-regulate mIgD and increase CD38 expression as they enter the germline center (GC) reaction, GC are sites on intense B-cell proliferation, AICDA dependent Ig hypermutation and class-switch recombination, and affinity maturation.

FIG. 2 discloses a schematic diagram of trimeric HIV-1 Env with sites of epitopes for broadly neutralizing antibodies. FIG. 2 depicts an HIV Env trimer with broadly neutralizing targets showing the need for a polyclonal multi-B lineage response to the CD4 binding site bnAb epitope and to at least two other epitopes such as the V1V2 glycan, the V3 glycan, fusion domain or the membrane proximal external region sites for a fully protective broadly neutralizing antibody response.

FIG. 3-1 shows MPER designs FIG. 3-1 discloses SEQ ID NOS 1-2, respectively, in order of appearance.

FIG. 3-2 shows high-throughput screening of MPER expression and antigenicity in 293-F cell transient transfection by flow cytometry.

FIG. 3-3 shows heatmap of MPER antibody binding to cell surface-expressed MPER constructs.

FIG. 3-4 shows heatmap of MPER antibody binding to mRNA-encoded cell surface-expressed MPER constructs.

FIG. 3-5 shows antibody binding to HV1303006 (B.MPER-TMD Y712I) measured by flow cytometry.

FIG. 3-6 shows flow cytometry histograms of antibody binding to HV1303006 (B.MPER-TMD Y712I).

FIG. 3-7 shows antibody binding to HV1303009 (GTH2-B.MPER-TMD) measured by flow cytometry.

FIG. 3-8 shows flow cytometry histogram of HV1303009 (GTH-B.MPER-TMD) binding to selected antibodies.

FIG. 3-9 shows antibody binding to HV1302976 (B.ConMPER-TMD-CD Y712I) measured by flow cytometry.

FIG. 3-10 shows flow cytometry histogram of HV1302976 (B.ConMPER-TMD-CD Y712I) binding to selected antibodies.

FIG. 4 depicts exemplary MPER peptide or HIV-1 envelope protein comprising a MPER peptide sequences (SEQ ID NOS 16-107, respectively, in order of appearance).

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

For the past 25 years, the HIV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30: 423, 2012) in which the induction of bnAb lineages is recreated. It was recently demonstrated the power of mapping the co-evolution of bnAbs and founder virus for elucidating the Env evolution pathways that lead to bnAb induction (Nature 496: 469, 2013).

### HIV-1 MPER Designs

Described herein are nucleic acid and amino acids sequences of HIV-1 MPERs or HIV-1 envelope proteins comprising a MPER peptide. The sequences for use as immunogens are in any suitable form. In certain embodiments, the described HIV-1 envelope sequences are gp160s. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

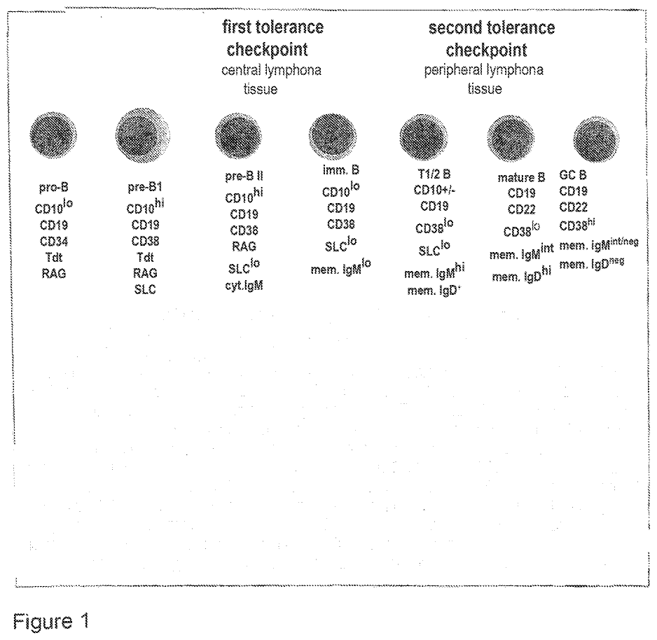

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR” (SEQ ID NO: 3). See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleave efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. April; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR (SEQ ID NO: 4) is changed to ERVVEREKE (SEQ ID NO: 5), and is one example of an uncleaved gp140 form. Another example is the gp140C form which has the REKR site (SEQ ID NO: 3) changed to SEKS (SEQ ID NO: 6). See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (rest of envelope sequence is indicated as “ . . . ”) (SEQ ID NO: 7). In other embodiments, the delta N-design described for CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See U.S. Pat. No. 10,040,826, e.g. at pages 10-12, the contents of which is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

The present invention relates in general, to a composition suitable for use in inducing anti-HIV-1 antibodies, and, in particular, to immunogenic compositions comprising mRNAs encoding HIV-1 membrane proximal external region (MPER) peptides to induce cross-reactive neutralizing antibodies and increase their breadth of coverage. In preferred embodiments, the composition comprises a nucleic acid encoding an MPER peptide.

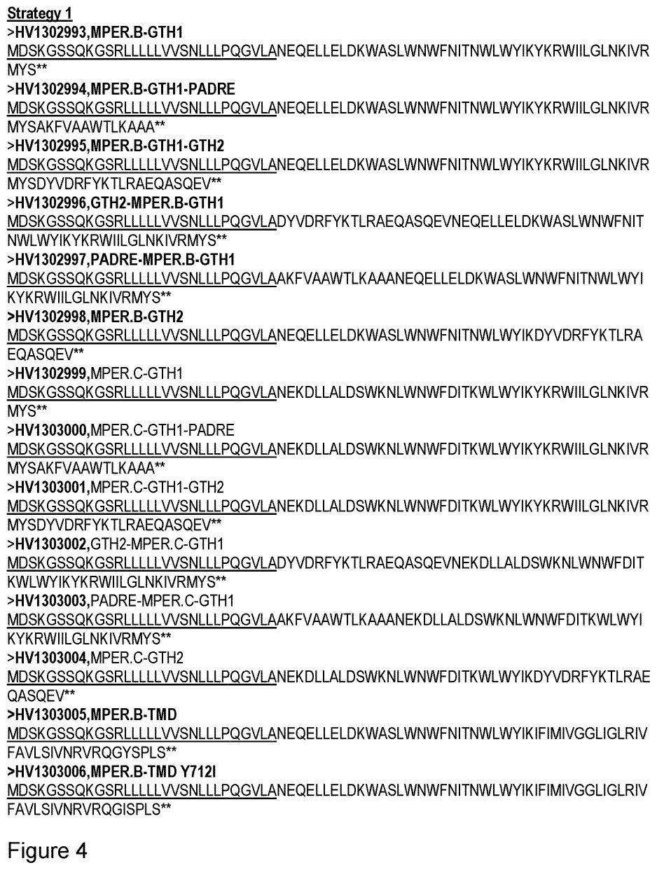

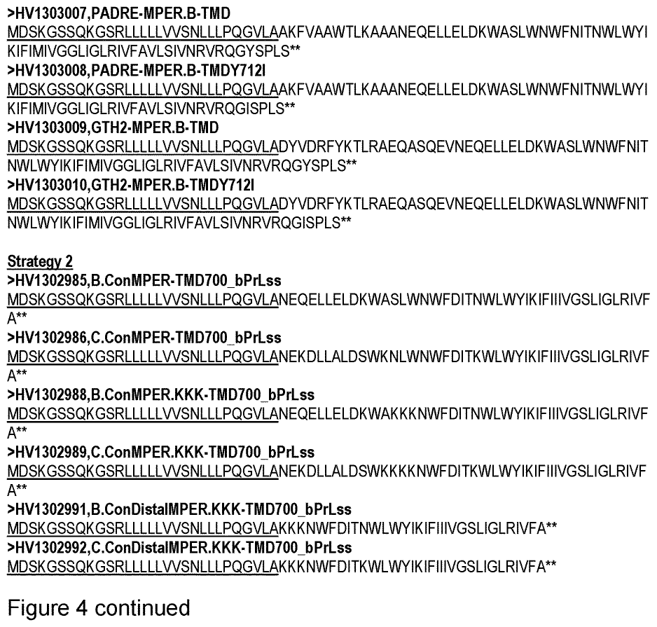

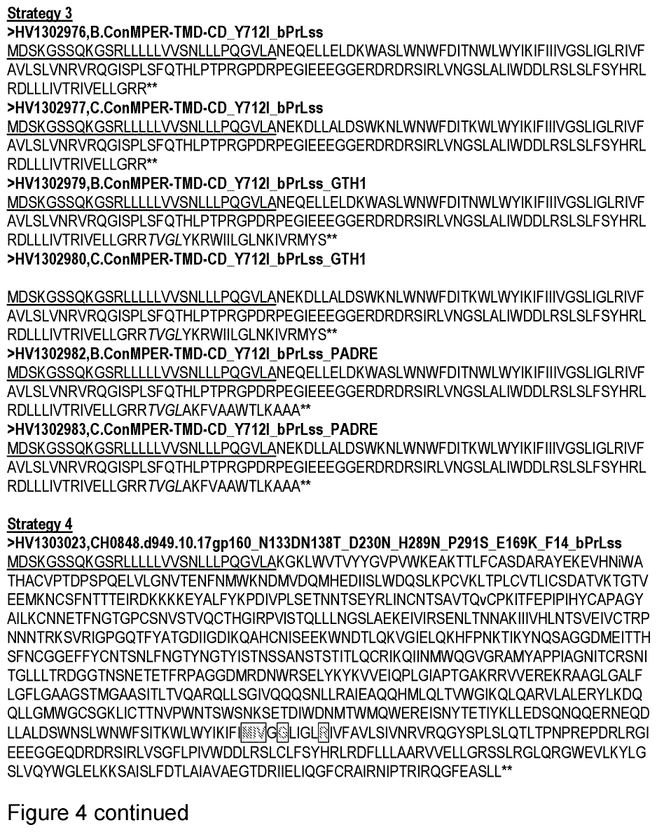

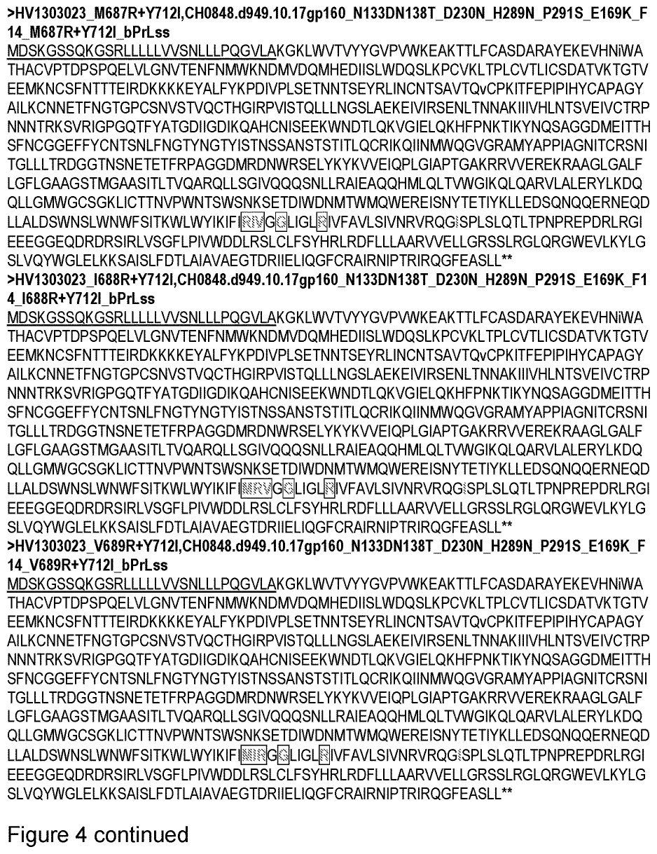

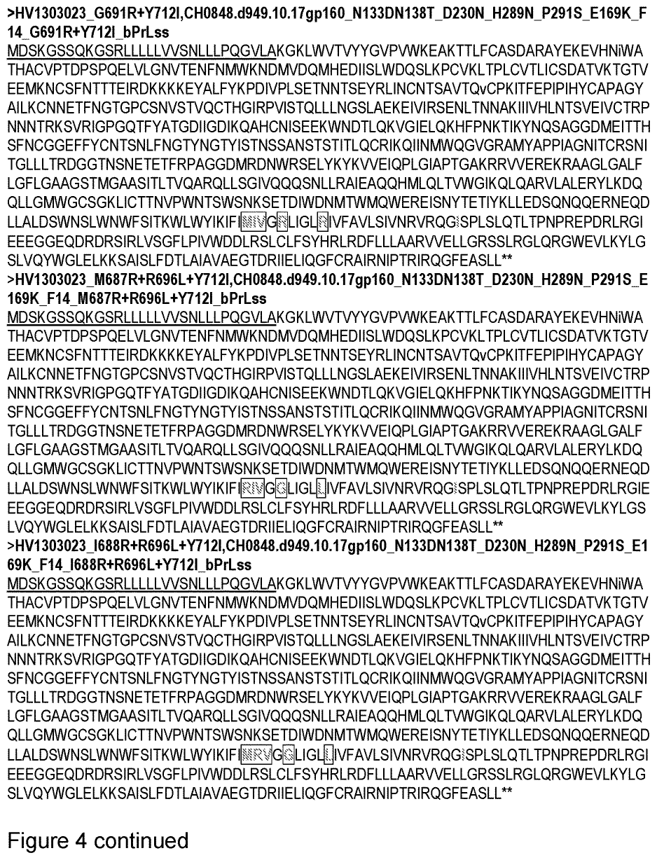

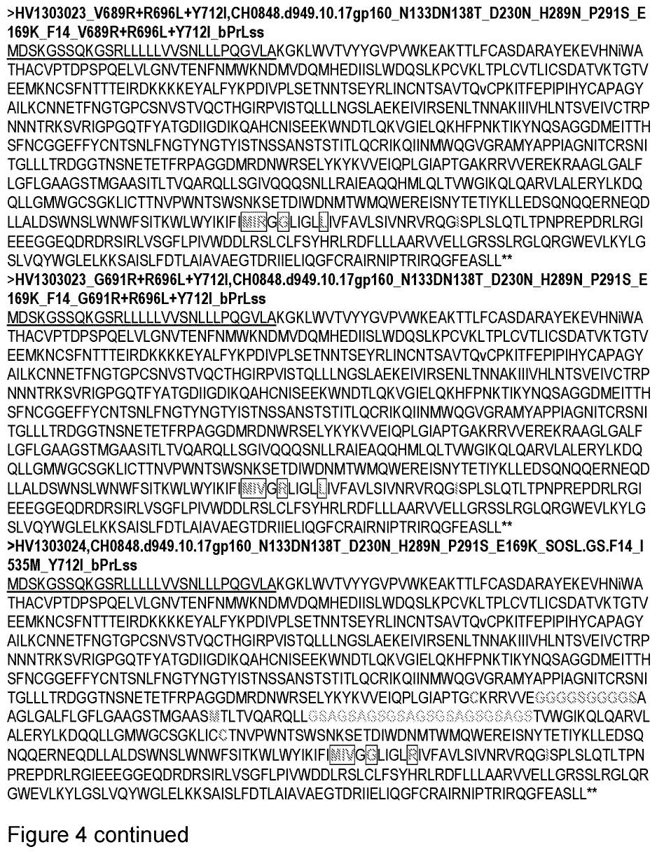

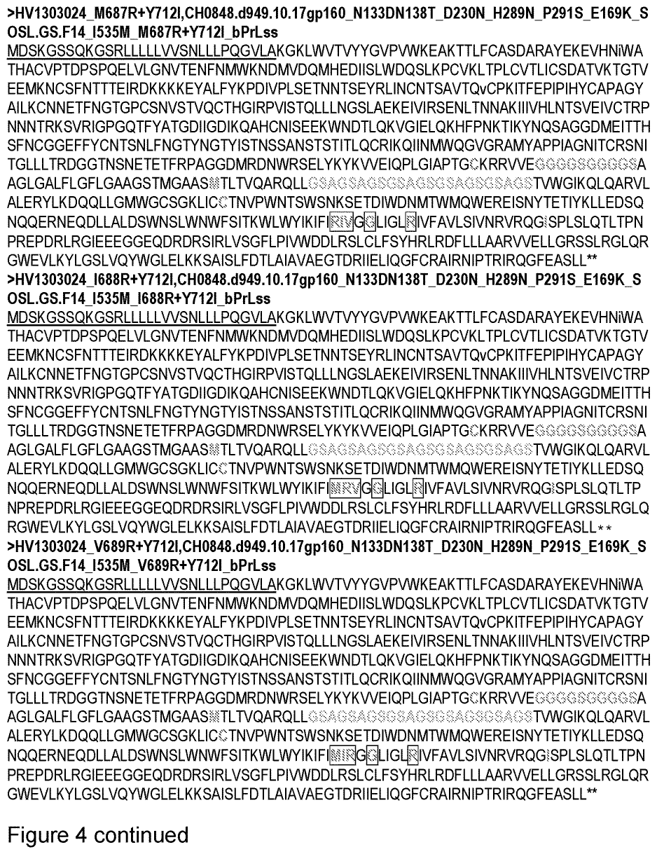

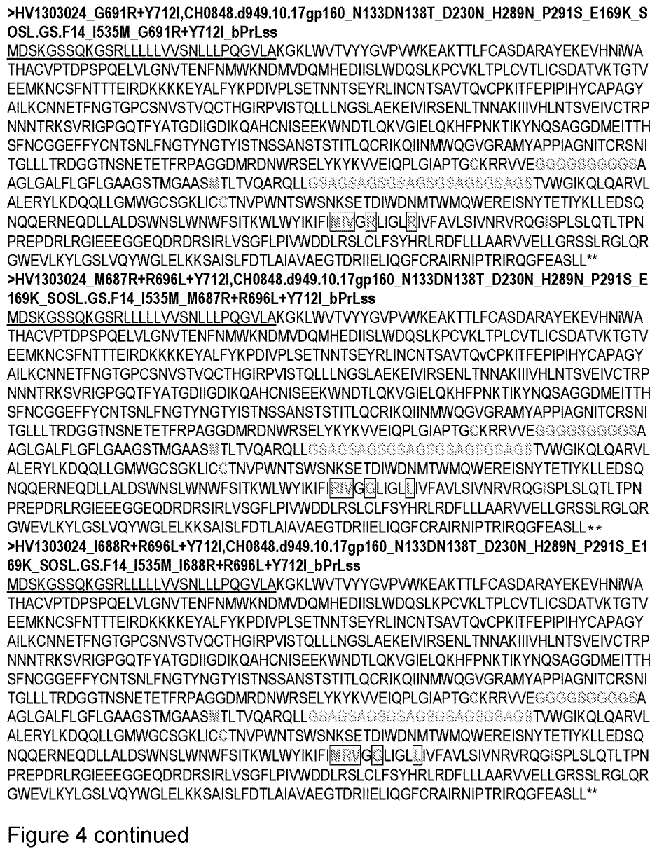

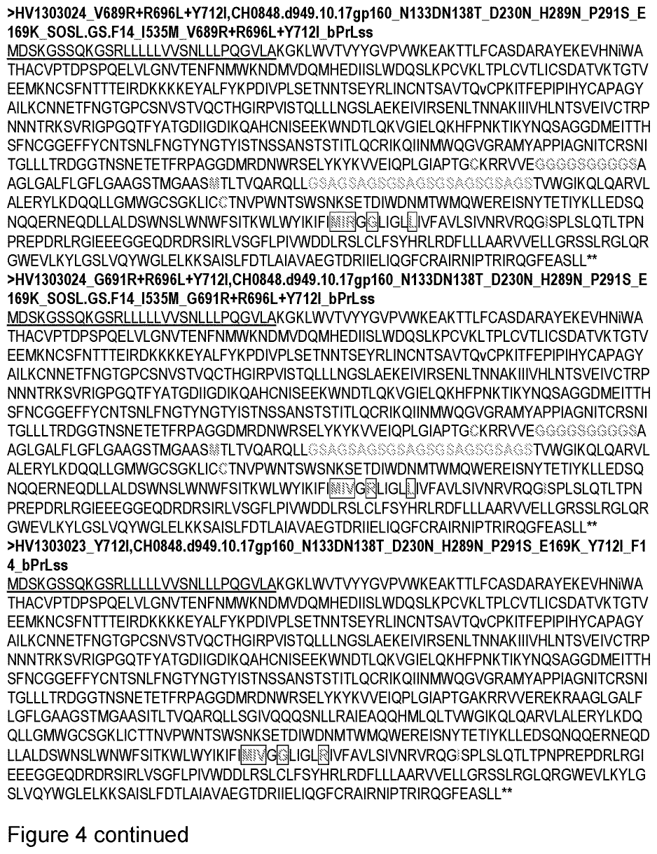

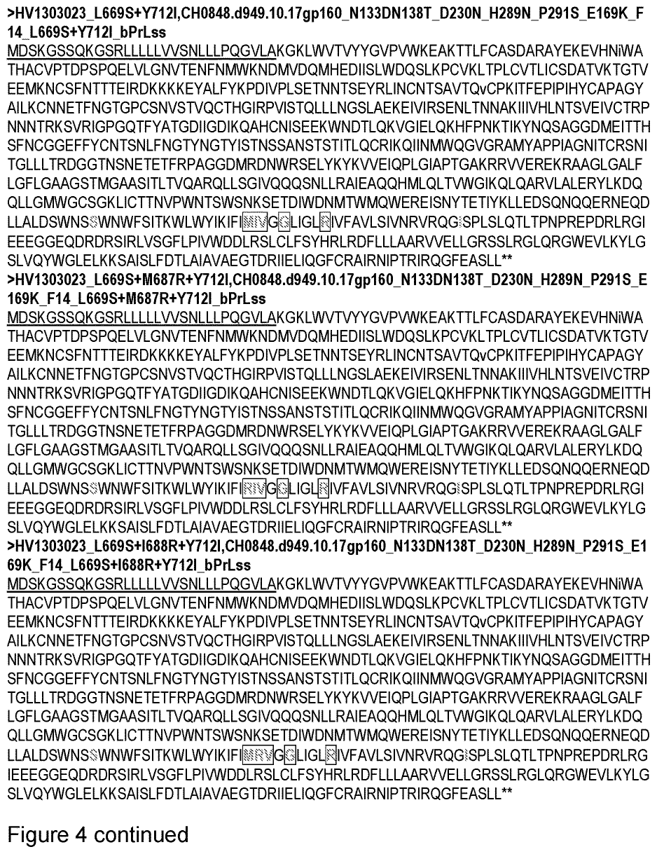

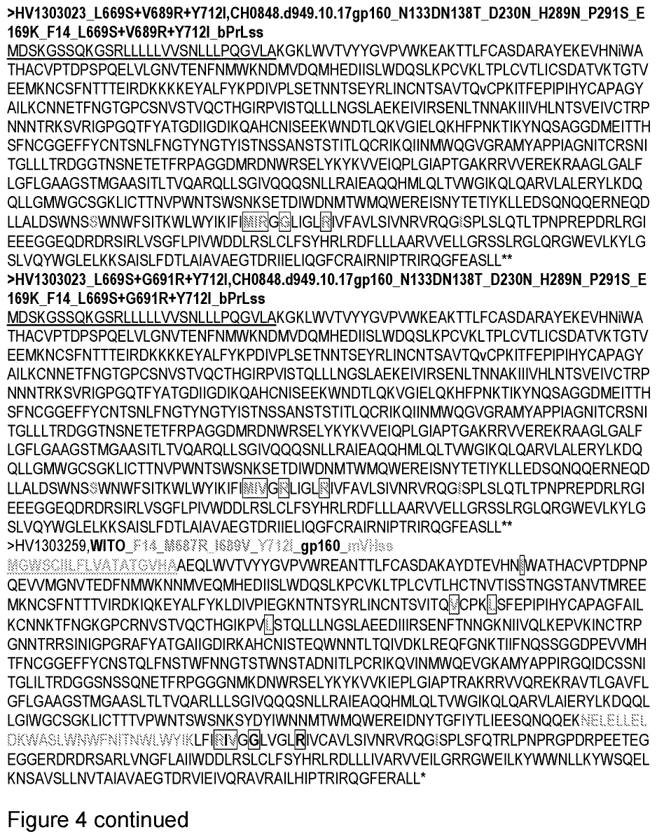

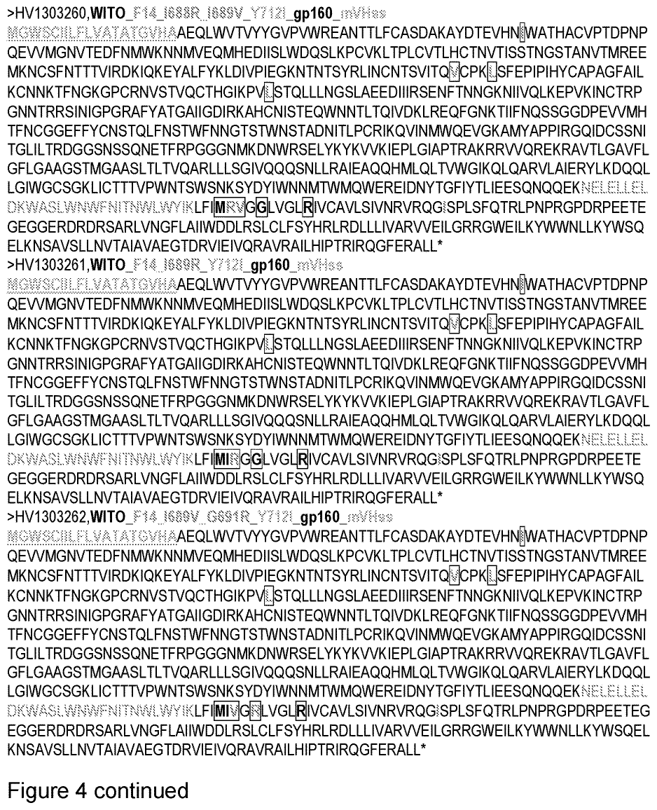

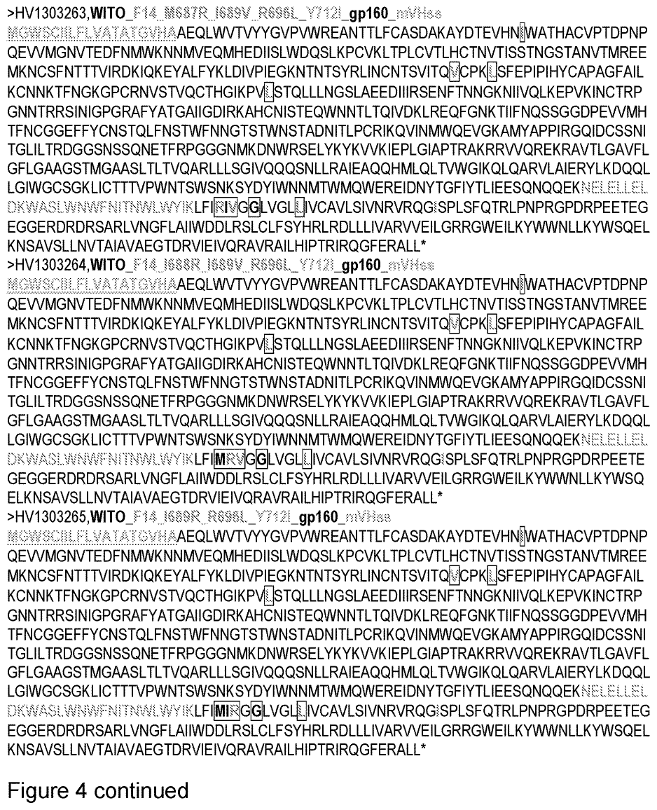

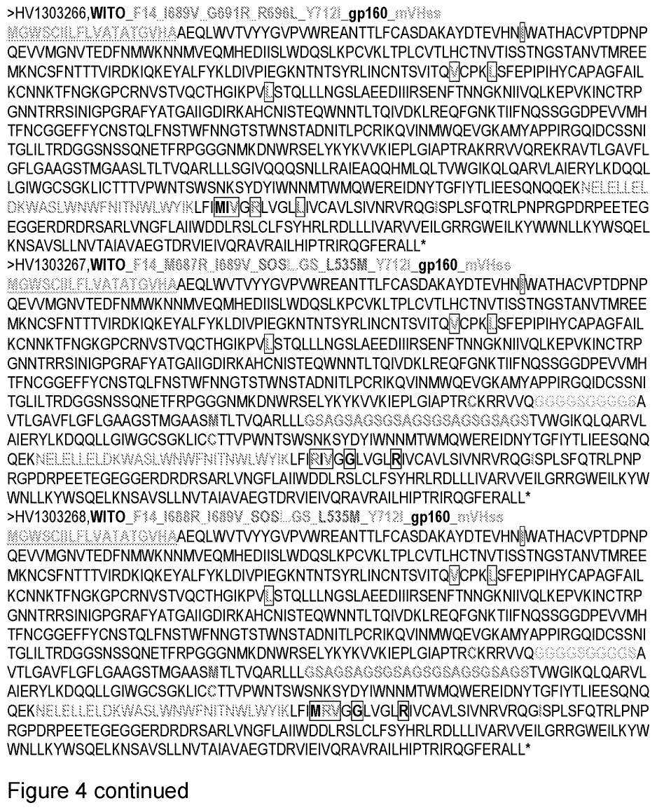

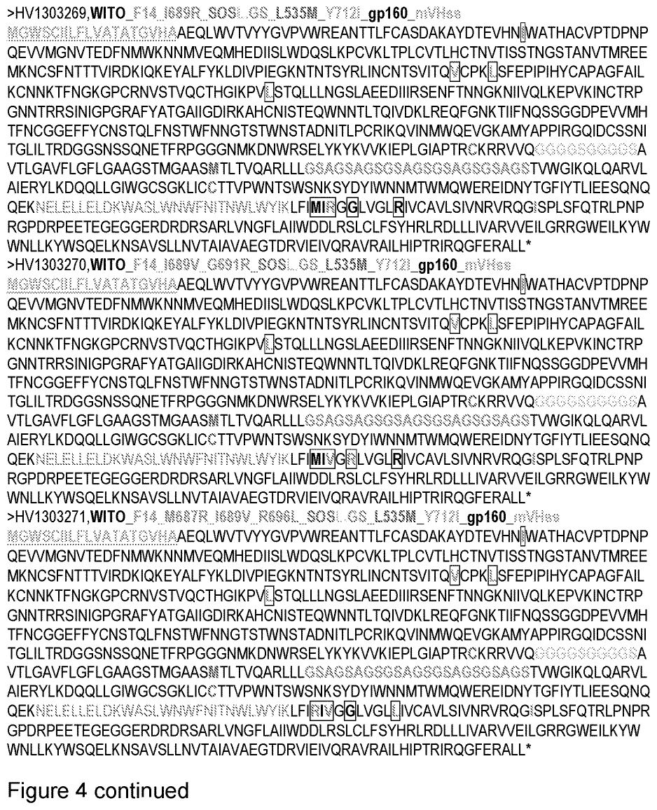

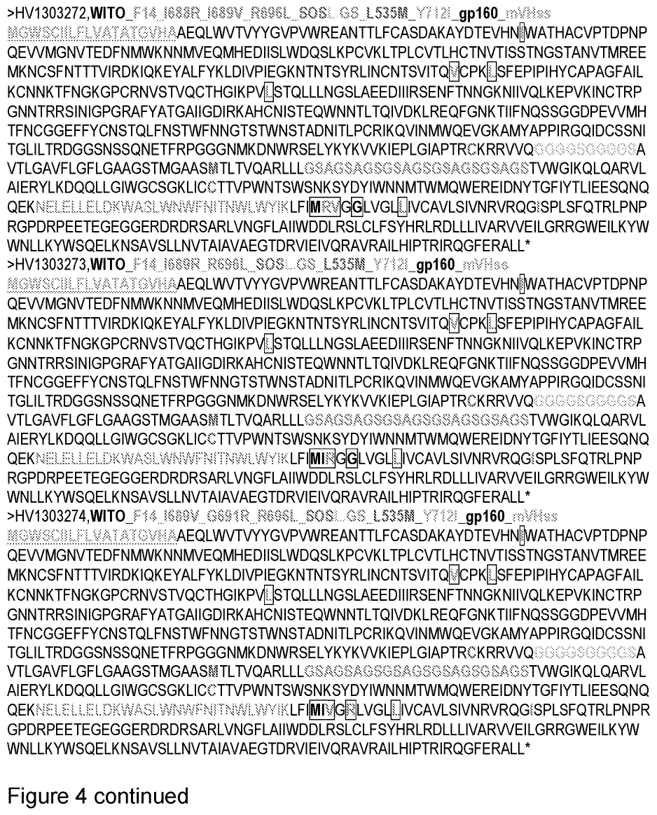

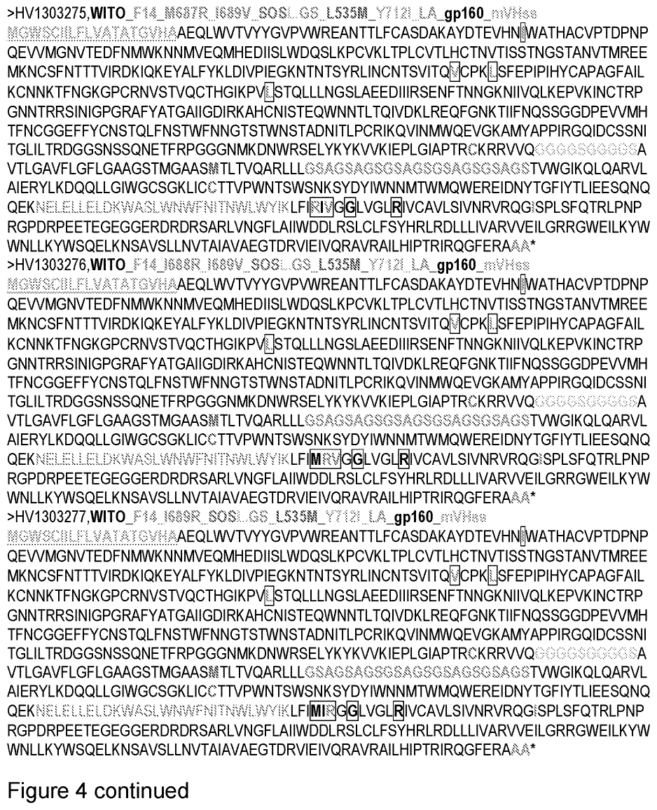

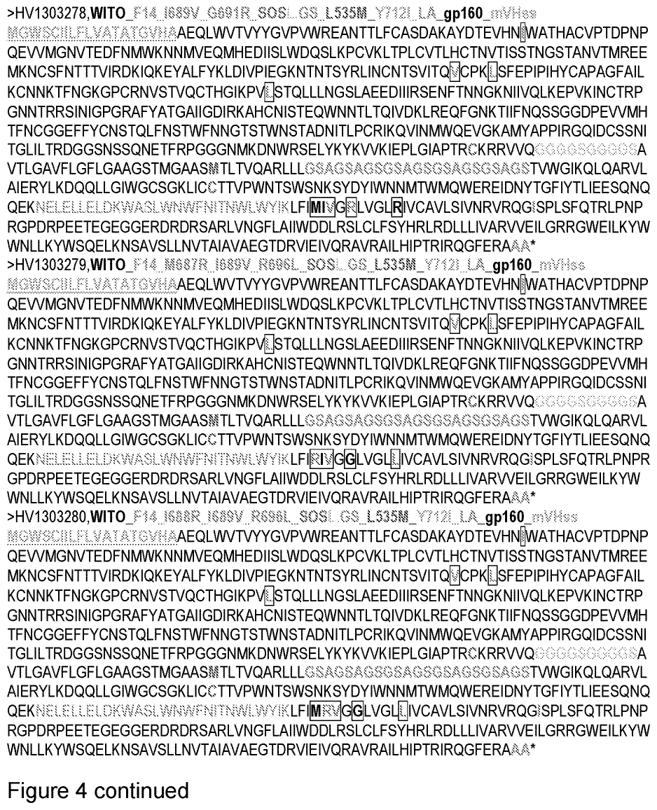

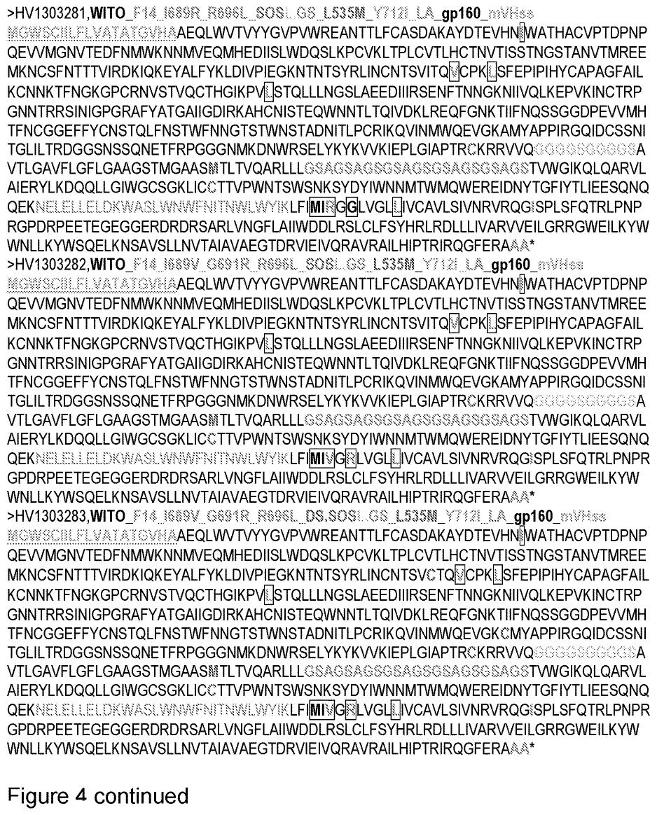

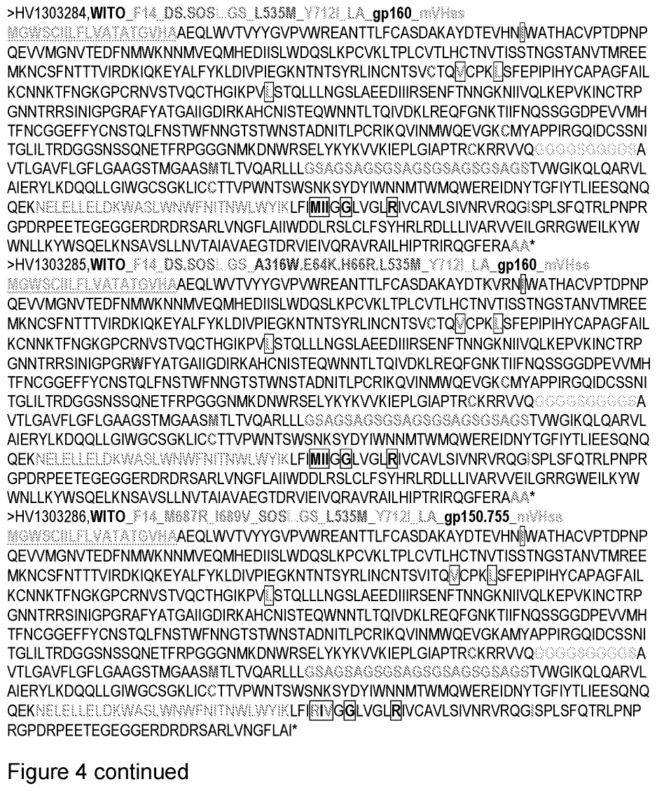

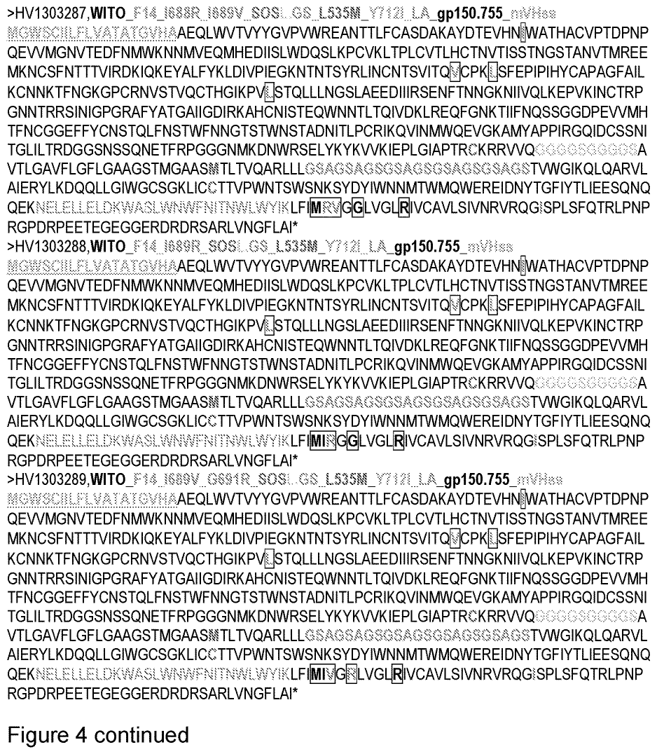

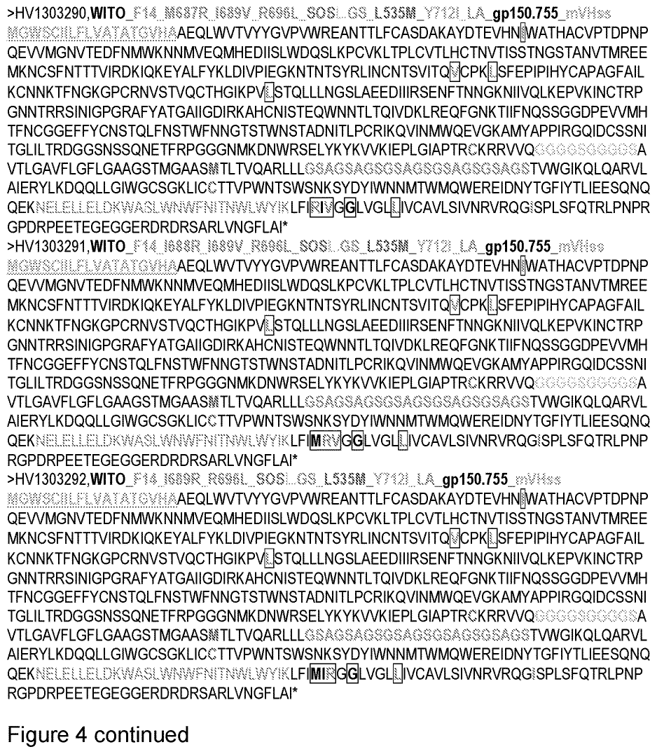

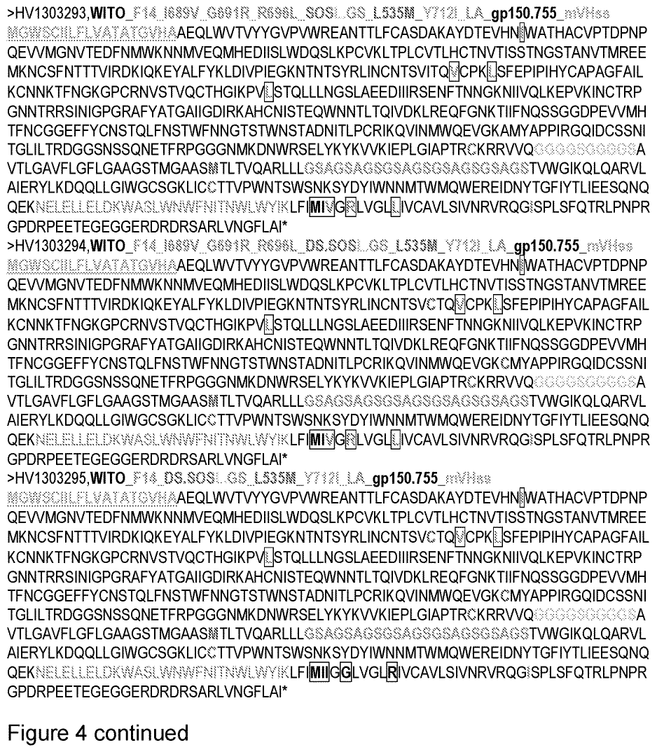

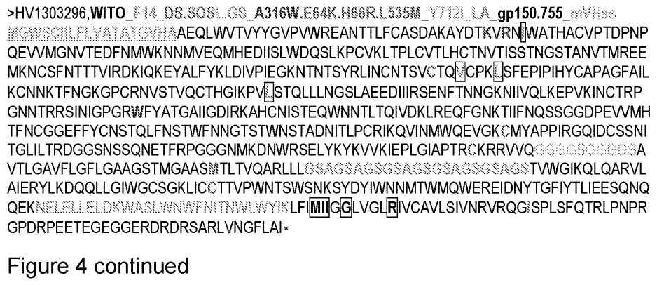

Suitable neutralizing antigens include gp41 MPER epitope peptides (Armbruster et al, J. Antimicrob. Chemother. 54:915-920 (2004), Stiegler and Katinger, J. Antimicrob. Chemother. 512:757-759 (2003), Zwick et al, Journal of Virology 79:1252-1261 (2005), Purtscher et al, AIDS10:587 (1996)) and variants thereof, for example, variants that confer higher neutralization sensitivity to MPER Mabs 2F5 and 4E10. In certain embodiments, the variant is a MPER epitope peptide with an L669S mutation that confers higher neutralization sensitivity to MPER mAbs 2F5 and 4E10 (Shen et al, J. Virology 83: 3617-25 (2009)). Exemplary suitable MPER peptides and HIV-1 envelope proteins comprising a MPER peptide are recited in FIG. 4.

The peptides, proteins, and/or nucleic acid immunogens of the invention can be formulated with, and/or administered with, adjuvants such as lipid A, oCpGs, TLR4 agonists or TLR 7 agonists that facilitate robust antibody responses (Rao et al, Immunobiol. Cell Biol. 82(5):523 (2004)). Non-limiting examples of other adjuvants that can be used include alum and Q521 (which do not break existing B cell tolerance). In certain embodiments, formulations comprise an adjuvant that is designed to break forms of B cell tolerance, such as oCpGs in an oil emulsion such as Emulsigen (an oil in water emulsion) (Tran et al, Clin. Immunol. 109(3):278-287 (2003)).

Autoreactive B cells can be activated by TLR ligands through a mechanism dependent on dual engagement of the B cell receptor (BCR) and TLR (Leadbetter et al, Nature 416:603 (2002); Marshak-Rothstein et al, Annu. Rev. Immunol. 25: 419-41 (2007), Herlands et al, Immunity 29:249-260 (2008), Schlomchik, Immunity 28:18-28 (2008)). In certain embodiments of immunogen design of the instant invention, soluble IFN-α is encapsulated into liposomes comprising MPER peptides such as MPER656 or MPER656-L669S peptides. IFN-α has been reported to modulate and relax the selectivity for autoreactive B cells by lowering the BCR activation threshold (Uccellini et al, J. Immunol. 181:5875-5884 (2008)). The design of the immunogens results from the observation that lipid reactivity of gp41MPER antibodies is required for both binding to membrane bound MPER epitopes and in the neutralization of HIV-1.

The B cell subsets that the liposomes can target include any B cell subset capable of making polyreactive antibodies that react with the epitopes of the MPER. These B cell subsets include, but are not limited to, the marginal zone IgM+ CD27+ B cell subset (Weill et al, Annu. Rev. Immunol. 27:267-85 (2009), Li et al, J. Exp. Med 195: 181-188 (2002)), the transitional populations of human B cells (Sims et al, Blood 105:4390-4398 (2005)), and the human equivalent of the B cells that express the human equivalent of the mouse Immunoglobulin (Ig) light chain lambda X (Li et al, Proc. Natl. Acad. Sci. 103:11264-11269 (2006), Witsch et al, J. Exp. Med. 203:1761-1772 (2006)). All of these B cell subsets have the capacity to make multi-reactive antibodies and, therefore, to make antibodies that have the characteristic of reacting with both lipids and HIV-1 gp41. Other antibodies may be multi-reactive and react to different epitopes on the same MPER peptide, different epitopes on two different MPER peptides, an epitope of an MPER peptide and another epitope.

The peptide, proteins and/or nucleic acid immunogens can be administered, for example, IV, intranasally, subcutaneously, intraperitoneally, intravaginally, or intrarectally. The route of administration can vary, for example, with the patient, the conjugate and/or the effect sought, likewise the dosing regimen.

### Nucleic Acid Sequences

In certain aspects the invention provides compositions and methods of MPER genetic immunization either alone or with MPER peptides or HIV-1 envelope proteins comprising a MPER peptide to recreate the swarms of evolved viruses that have led to bnAb induction. Nucleotide-based vaccines offer a flexible vector format to immunize against virtually any protein antigen. Currently, two types of genetic vaccination are available for testing-DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M. smegmatis) (Yu, J S et al. Clinical Vaccine Immunol. 14: 886-093, 2007; ibid 13: 1204-11, 2006), and recombinant vaccinia type of vectors (Santra S. Nature Med. 16: 324-8, 2010), for example but not limited to ALVAC, replicating (Kibler K V et al., PLoS One 6: e25674, 2011 nov 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by In-Cell-Art.

In certain aspects, the invention provides nucleic acids comprising sequences encoding MPER peptides or HIV-1 envelope proteins comprising a MPER peptide of the invention. In certain embodiments, the nucleic acids are DNAs. In certain aspects, the invention provides expression vectors comprising the nucleic acids of the invention.

In certain aspects the invention provides nucleic acids encoding the inventive MPER peptides or HIV-1 envelope proteins comprising a MPER peptide (e.g., any one of the MPER peptides or MPER peptides or HIV-1 envelope proteins comprising a MPER peptide disclosed in FIG. 4).

In certain embodiments the nucleic acid encoding an MPER peptide (e.g., any one of the MPER peptides or HIV-1 envelope proteins comprising a MPER peptide disclosed in FIG. 4) is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector. In some embodiments, the expression vector comprises DNA.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

The recombinant nucleic acid sequence can be an optimized nucleic acid sequence. Such optimization can increase or alter the immunogenicity of the envelope. Optimization can also improve transcription and/or translation. Optimization can include one or more of the following: low GC content leader sequence to increase transcription; mRNA stability and codon optimization; addition of a Kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for expression and immunogenicity determination of nucleic acid encoded MPER peptides are known in the art.

In certain aspects the invention contemplates using immunogenic compositions wherein immunogens are delivered as recombinant proteins. In certain embodiments recombinant proteins are produced in CHO cells.

The immunogenic MPER peptides or HIV-1 envelope proteins comprising a MPER peptide can also be administered as a protein boost in combination with a variety of nucleic acid MPER primes (e.g., HIV-1 MPER peptides or HIV-1 envelope proteins comprising a MPER peptide delivered as DNA expressed in viral or bacterial vectors).

Dosing of proteins and nucleic acids can be readily determined by a skilled artisan. A single dose of nucleic acid can range from a few nanograms (ng) to hundreds of micrograms (g) or milligram of a single immunogenic nucleic acid. Recombinant protein dose can range from a few g micrograms to a few hundred micrograms, or milligrams of a single immunogenic polypeptide.

Administration: The compositions can be formulated in designs that incorporate appropriate carriers such as peptides for enhancing CD4+ T cell help, known as PADRE, GTH1, GTH2, or any combination thereof. In certain embodiments the compositions are delivered via intramuscular (IM), via subcutaneous, via intravenous, via nasal, via mucosal routes, or any other suitable route of immunization.

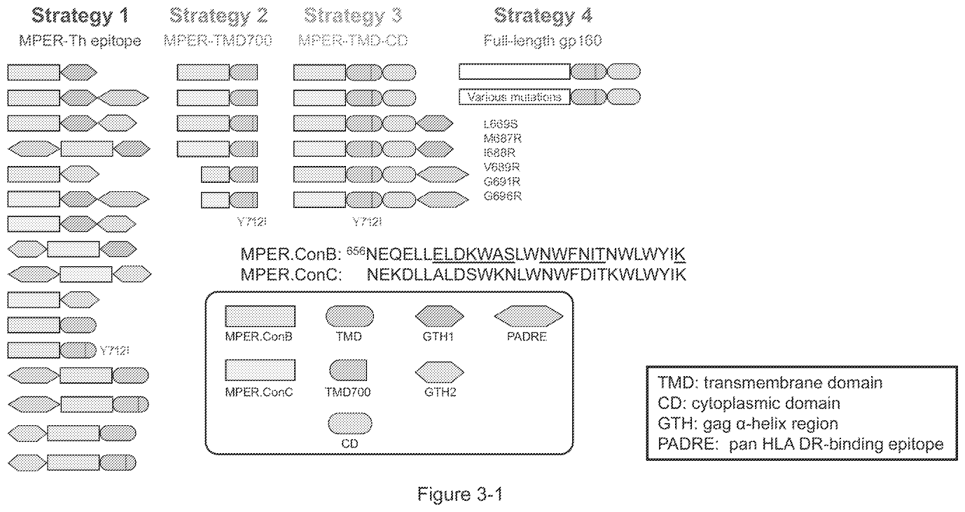

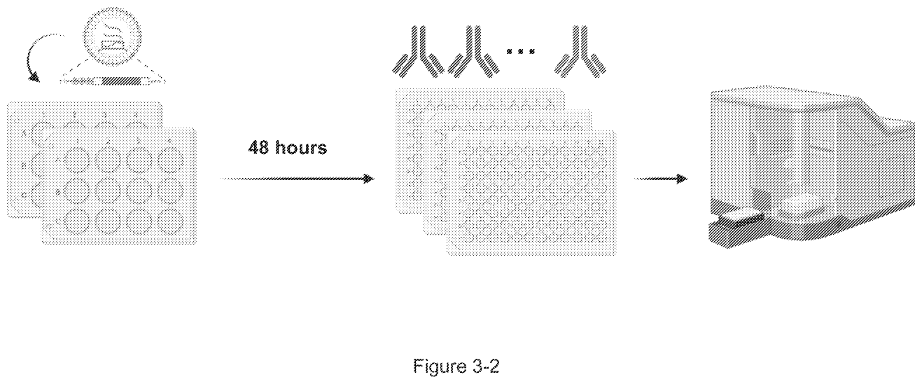

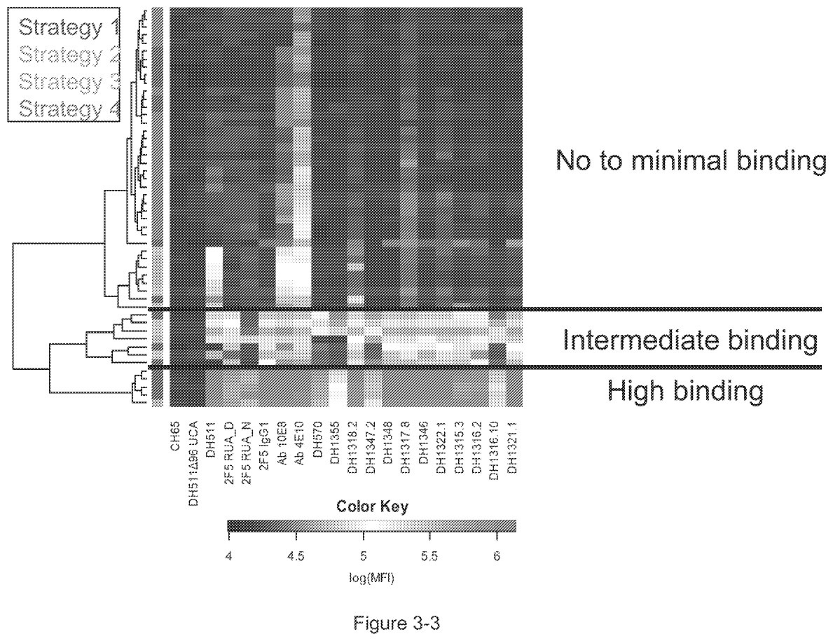

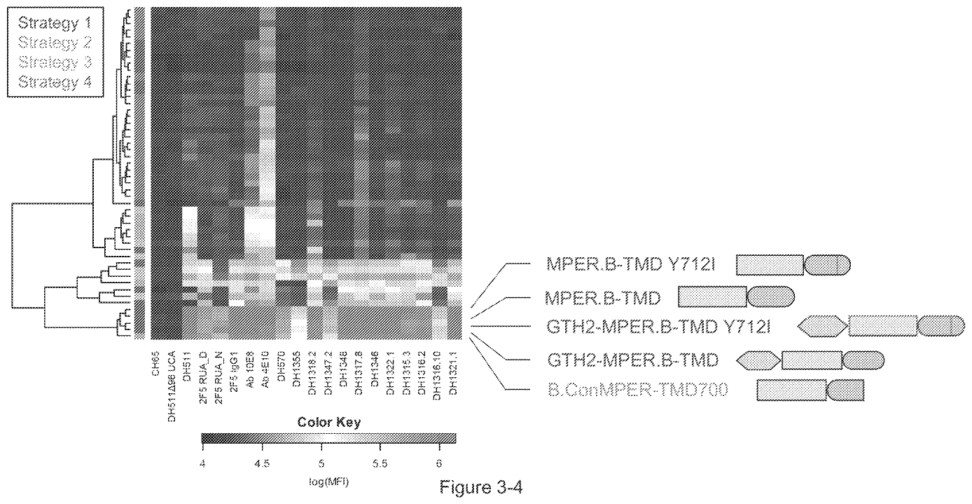

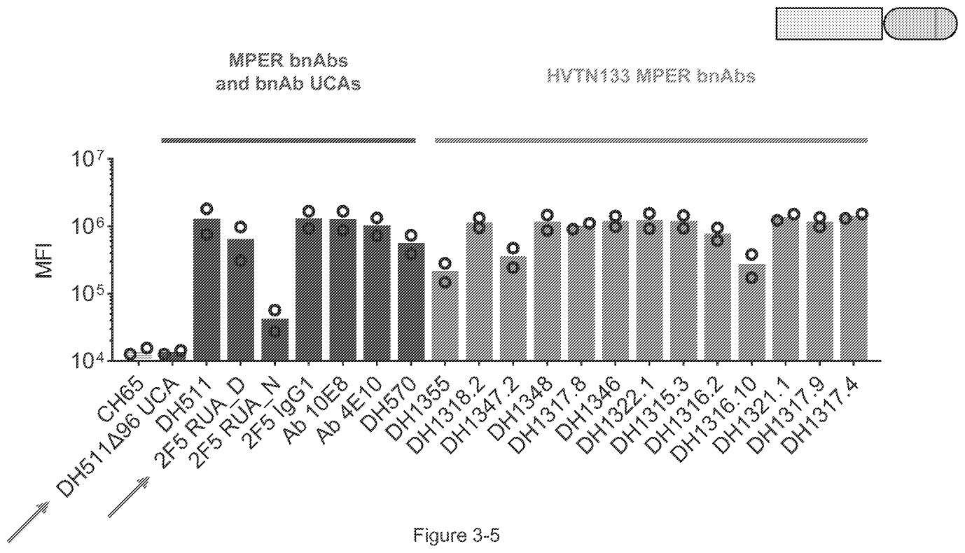

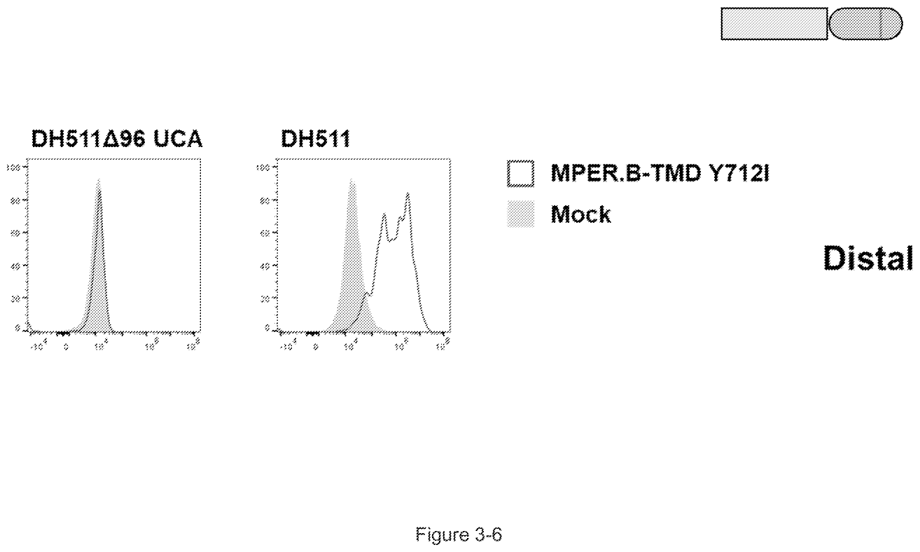

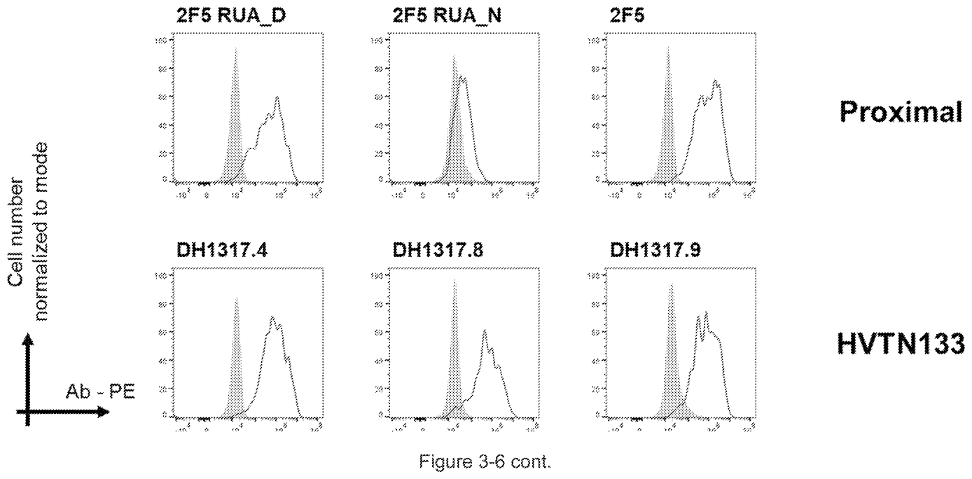

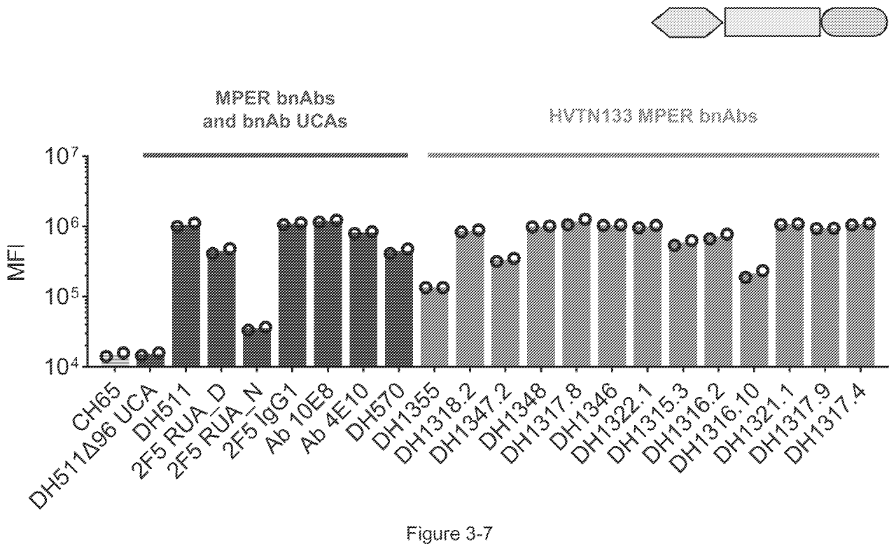

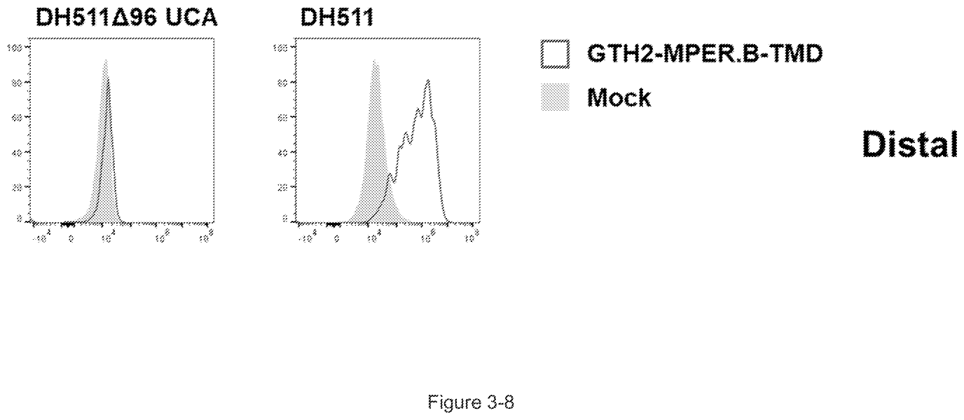

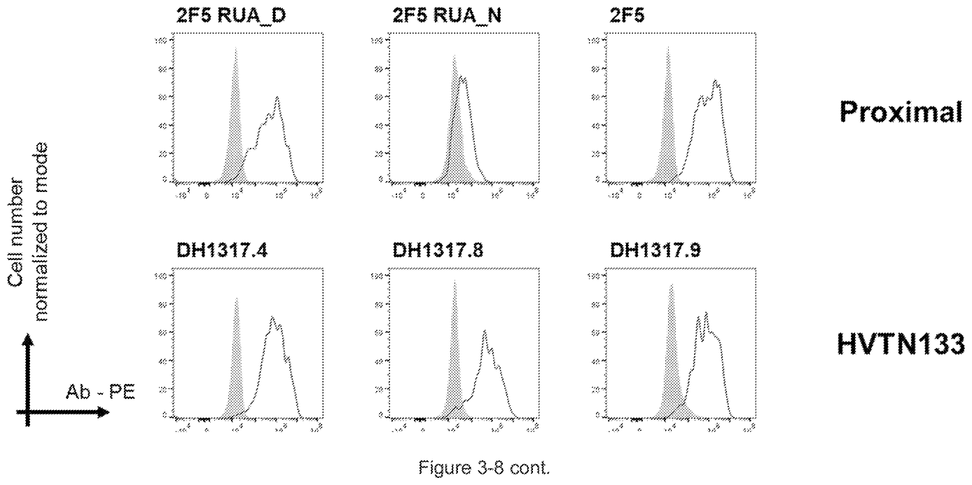

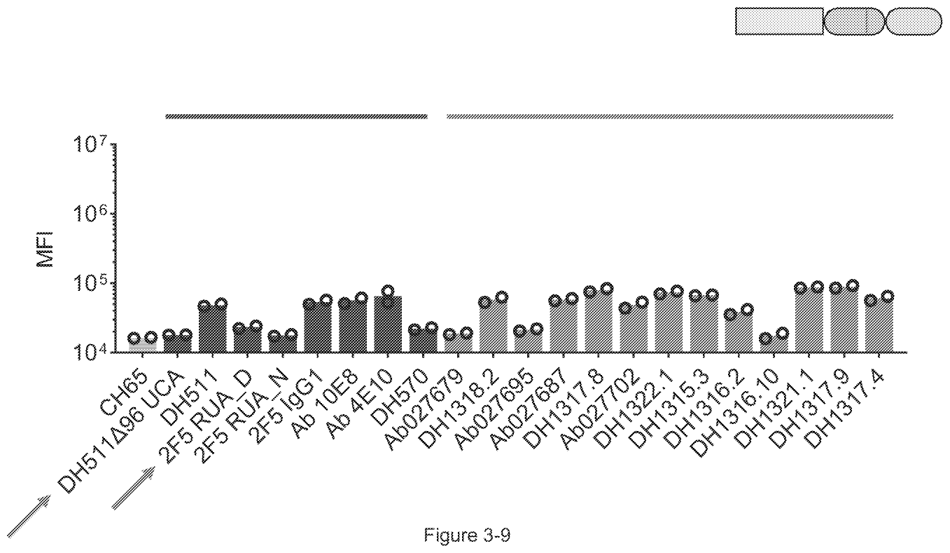

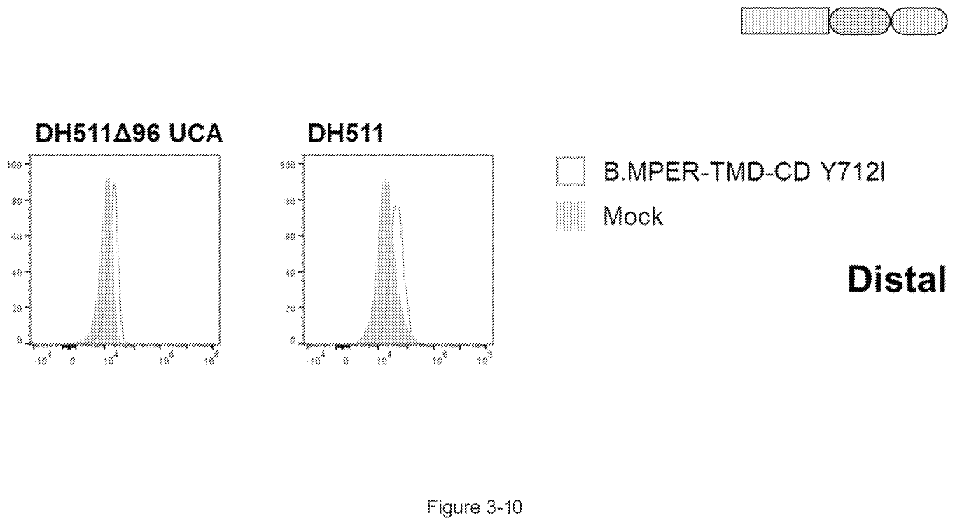

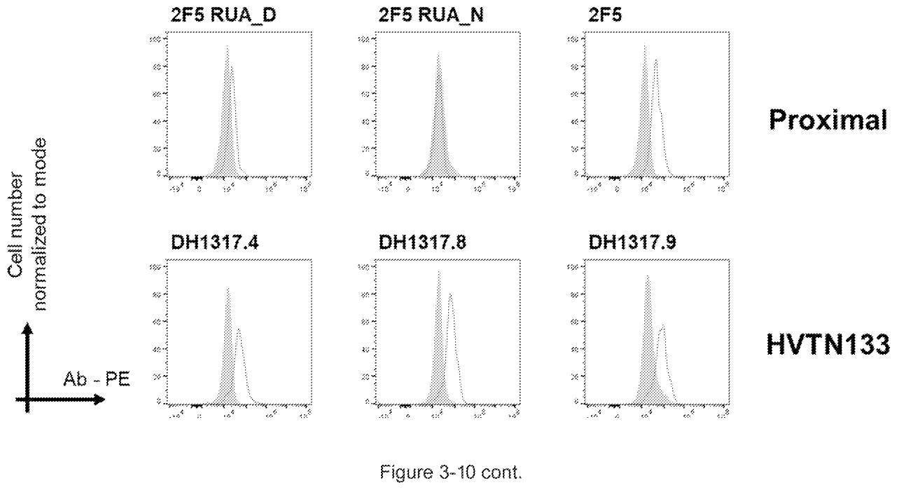

Some of the non-transmembrane compositions (e.g., in Strategy 1; see FIG. 3-1) can be expressed as proteins and formulated with appropriate carriers and adjuvants using techniques to yield compositions suitable for immunization. The compositions can include an adjuvant, such as, for example but not limited to, alum, 3M052, poly IC, MF-59 or other squalene-based adjuvant, ASOIB, or other liposomal based adjuvant suitable for protein or nucleic acid immunization. In certain embodiments, the adjuvant is GSK ASOlE adjuvant containing MPL and QS21. This adjuvant has been shown by GSK to be as potent as the similar adjuvant AS01B but to be less reactogenic using HBsAg as vaccine antigen (Leroux-Roels et al., IABS Conference, April 2013). In certain embodiments, TLR agonists are used as adjuvants. In other embodiment, adjuvants which break immune tolerance are included in the immunogenic compositions. The compositions with transmembrane domains are encoded by nucleic acids such as messenger RNAs (mRNAs) or DNAs (e.g., plasmids).

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355|Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody. Non-limiting examples are ipilimumab and tremelimumab. In certain embodiments, the methods comprise administering a second immunomodulatory agent, wherein the second and first immunomodulatory agents are different.

There are various host mechanisms that control bnAbs. For example, highly somatically mutated antibodies become autoreactive and/or less fit (Immunity 8: 751, 1998; PloS Comp. Biol. 6 e1000800, 2010; J. Thoret. Biol. 164:37, 1993); Polyreactive/autoreactive naïve B cell receptors (unmutated common ancestors of clonal lineages) can lead to deletion of Ab precursors (Nature 373: 252, 1995; PNAS 107: 181, 2010; J. Immunol. 187: 3785, 2011); Abs with long HCDR3 can be limited by tolerance deletion (JI 162: 6060, 1999; JCI 108: 879, 2001). BnAb knock-in mouse models are providing insights into the various mechanisms of tolerance control of MPER BnAb induction (deletion, anergy, receptor editing). Other variations of tolerance control likely will be operative in limiting BnAbs with long HCDR3s, high levels of somatic hypermutations.

It is readily understood that the MPER peptides or HIV-1 envelope proteins comprising a MPER peptide comprise a signal peptide/leader sequence. It is well known in the art that HIV-1 envelope polypeptide is a secretory protein with a signal peptide or leader sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal peptide sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA (SEQ ID NO: 12)). Most of the chimeric designs include CD5 leader sequence. A skilled artisan appreciates that when used as immunogens, and for example when recombinantly produced, the amino acid sequences of these proteins (e.g., MPER peptides or HIV-1 envelope proteins comprising a MPER peptide) do not comprise the signal peptide/leader sequences.

Any one of the MPERs or a nucleic acid encoding any one of the MPER peptides or HIV-1 envelope proteins comprising a MPER peptide of the invention could be designed and expressed as described herein.

### HIV-1 Envelope Trimers and Other Envelope Designs

Elicitation of neutralizing antibodies is one goal for antibody-based vaccines. Neutralizing antibodies target the native trimeric HIV-1 Env on the surface virions. The trimeric HIV-1 envelope protein consists of three protomers each containing a gp120 and gp41 heterodimer. Recent immunogen design efforts have generated soluble near-native mimics of the Env trimer that bind to neutralizing antibodies but not non-neutralizing antibodies. The recapitulation of the native trimer could be a key component of vaccine induction of neutralizing antibodies. Neutralizing Abs target the native trimeric HIV-1 Env on the surface of viruses (Poignard et al. J Virol. 2003 January; 77(1):353-65; Parren et al. J Virol. 1998 December; 72(12):10270-4.; Yang et al. J Virol. 2006 November; 80(22):11404-8.). The HIV-1 Env protein consists of three protomers of gp120 and gp41 heterodimers that are noncovalently linked together (Center et al. J Virol. 2002 August; 76(15):7863-7.). Soluble near-native trimers preferentially bind neutralizing antibodies as opposed to non-neutralizing antibodies (Sanders et al. PLoS Pathog. 2013 September; 9(9): e1003618).

In certain aspects, the MPER peptides described herein can be incorporated into any HIV-1 envelope sequence. In certain aspects, the MPER peptides described herein can be incorporated into any HIV-1 envelope sequence from the CH848 infected individual and variants thereof. See e.g., US2020/0113997 incorporated herein by reference in its entirety including FIGS. 40A-C, 41A-41C, 44A-D, 45, 46, 47A, 49A-B, 50A-D, 51, 52A-B, 53A, 53D, 54A-F, 77A-L, and 78A-B and SEQ ID NOs disclosed therein. In some embodiments, the MPER peptides can be incorporated into envelope CH848.3.D0949.10.17 (also referred to as CH848.d0949.10.17WT; see US2020/0113997 incorporated herein by reference in its entirety including FIGS. 39A-B and SEQ ID NOs disclosed therein) and variants thereof, including, but not limited to, CH848.d0949.10.17 DT (also referred to as CH848.d0949.10.17.N133D.N138T; see US2020/0113997 incorporated herein by reference in its entirety including FIG. 49A, 50B, 51, 52A, 53D, 54A-C, 77D-G, 771 and SEQ ID NOs disclosed therein). In certain aspects the invention provides a recombinant HIV-1 envelope that in addition to comprising the MPER peptides described herein can further lack glycosylation at position N133 and N138 (HXB2 numbering), comprise glycosylation at N301 (HXB2 numbering) and N332 (HXB2 numbering), comprise modifications wherein glycan holes are filled (D230N_H289N_P291S (HXB2 numbering)), comprise the “GDIR” or “GDIK” motif, or any trimer stabilization modifications, UCA targeting modification, immunogenicity modification, F14 and/or VT8 modifications, or combinations thereof. In certain embodiments the recombinant envelope optionally comprises any combinations of these modifications.

In certain embodiments the MPER peptides described herein can be incorporated into any HIV-1 envelope sequence comprising E169K (HXB2 numbering). In certain embodiments, CH848.d0949.10.17DT envelope comprises additional modifications D230N.H289N.P291S.E169K and is referred to as CH848.d0949.10.17 Dte. In certain embodiments, CH848.d0949.10.17 envelope comprises additional modifications D230N.H289N.P291S.E169K and is referred to as CH848.d0949.10.17WTe. In certain embodiments, CH848.d0949.10.17DT envelope comprises additional modifications referred to as CH848.0949.10.17DT.GS designs. In certain embodiments, CH848.d0949.10.17DT.GS envelopes comprise additional modifications D230N.H289N.P291S.E169K.

The invention contemplates any other design, e.g. stabilized trimer, of the sequences described here in. For non-limiting embodiments of additional stabilized trimers see US2015/0366961, US2020/0002383, US2021/0187091 and US2020/0113997, F14 and/or VT8 designs (US2021/0379177) all of which are incorporated by reference in their entirety.

Multimeric nanoparticles that comprise and/or display HIV envelope protein or fragments on their surface can be used a vaccine immunogens.

In some instances, the nucleic acid encoding an antigen (e.g., a HIV-1 envelope polypeptide comprising the MPER peptides described herein) is fused via a linker/spacer to a nucleic acids sequence encoding a protein which can self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

To improve the interaction between the naïve B cell receptor and immunogens, in some embodiments, an envelope design is created so the envelope is presented on particles, e.g. but not limited to nanoparticle. In some embodiments, the HIV-1 envelope trimer could be fused to ferritin. Ferritin protein self assembles into a small nanoparticle with three-fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 2015 12:82, DOI: 10.1186/s12977-015-0210-4.

Any suitable ferritin sequence could be used. In non-limiting embodiments, ferritin sequences are disclosed in U.S. Pat. No. 10,961,283, the content of which is hereby incorporated by reference in its entirety.

Ferritin nanoparticle linkers: The ability to form HIV-1 envelope ferritin nanoparticles relies self-assembly of 24 ferritin subunits into a single ferritin nanoparticle. The addition of a ferritin subunit to the C-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, ferritin can be designed with elongated glycine-serine linkers to further distance the envelope from the ferritin subunit. To make sure that the glycine linker is attached to ferritin at the correct position, constructs can be created that attach at second amino acid position or the fifth amino acid position. The first four n-terminal amino acids of natural Helicobacter pylori ferritin are not needed for nanoparticle formation but may be critical for proper folding and oligomerization when appended to envelope. Thus, constructs can be designed with and without the leucine, serine, and lysine amino acids following the glycine-serine linker. The goal will be to find a linker length that is suitable for formation of envelope nanoparticles when ferritin is appended to most envelopes. Any suitable linker between the envelope and ferritin could be used, so long as the fusion protein is expressed and the trimer is formed.

The nanoparticle immunogens are composed of various forms of HIV-1 envelope protein, e.g. without limitation envelope trimer, and self-assembling protein, e.g. without limitation ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the envelope protein on its surface.

Another approach to multimerize expression constructs uses staphylococcus sortase A transpeptidase ligation to conjugate inventive envelope trimers, for example but not limited to cholesterol. The trimers can then be embedded into liposomes via the conjugated cholesterol. To conjugate the trimer to cholesterol either a C-terminal LPXTG tag or a N-terminal pentaglycine repeat tag is added to the envelope trimer gene. Cholesterol is also synthesized with these two tags. In a non-limiting embodiment, a C-terminal tag is LPXTGG, where X signifies any amino acid but most commonly Ala, Ser, Glu. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles.

The invention provides designs of envelopes and trimer designs wherein the envelope comprises a linker which permits addition of a lipid, such as but not limited to cholesterol, via a sortase A reaction. See e.g., Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J. Org. Chem. 2007, 72, 3909-3912.

The lipid modified envelopes and trimers could be formulated as liposomes. Any suitable liposome composition is contemplated.

The lipid modified and multimerized envelopes and trimers could be formulated as liposomes. Any suitable liposome composition is contemplated.

Non-limiting embodiments of envelope designs for use in sortase A reaction are shown in FIG. 24 B-D of US2020/0002383, incorporated by reference in its entirety.

Additional sortase linkers could be used so long as their position allows multimerization of the envelopes. In a non-limiting embodiment, a C-terminal tag is LPXTG, where X signifies any amino acid but most commonly Ala, Ser, Glu, or a N-terminal pentaglycine repeat tag is added to the envelope trimer gene. In a non-limiting embodiment, a C-terminal tag is LPXTGG, where X signifies any amino acid but most commonly Ala, Ser, Glu.

### EXEMPLARY EMBODIMENTS

Provided are engineered MPER peptide immunogens derived from multiple viruses. Several strategies have been used to develop MPER peptides and encoding nucleic acids. See FIGS. 3-1 to 3-10.

Any modification or combination of the modifications described herein, including but not limited, to different versions of soluble proteins, different versions of membrane expressed proteins, stabilization mutations, furin cleavage site mutations, signal peptides, and/or cytoplasmic tail modifications can be applied to any MPER peptide sequence or HIV-1 envelope protein sequence comprising a MPER peptide.

In certain aspects, the invention provides a recombinant HIV-1 MPER peptide comprising all the consecutive amino acids after the signal peptide of SEQ ID NOs: 1, 2, or 16-45 or a HIV-1 envelope protein sequence comprising a MPER peptide comprising all the consecutive amino acids after the signal peptide of SEQ ID NOs: 46-107. In some embodiments, the recombinant HIV-1 MPER peptide comprises all the consecutive amino acids after the signal peptide of SEQ ID NO: 28, 29 or 40.

In certain aspects, the invention provides a nucleic acid comprising a sequence encoding the recombinant HIV-1 MPER peptide or HIV-1 envelope protein sequence comprising a MPER peptide described herein.

In certain aspects, the invention provides an immunogenic composition comprising the MPER peptide or HIV-1 envelope protein sequence comprising a MPER peptide described herein and a carrier.

In certain aspects, the invention provides an immunogenic composition comprising the nucleic acid described herein and a carrier.

In some embodiments, the immunogenic composition further comprises an adjuvant.

In some embodiments, the nucleic acid is operably linked to a promoter, and optionally the nucleic acid is inserted in an expression vector.

In certain aspects, the invention provides a method of inducing an immune response in a subject comprising administering a composition comprising any suitable form of a nucleic acid(s) described herein or the recombinant HIV-1 MPER peptide or HIV-1 envelope protein sequence comprising a MPER peptide described herein in an amount sufficient to induce an immune response. In some embodiments, the composition further comprises an adjuvant. In some embodiments, the method further comprises administering an agent which modulates host immune tolerance. In some embodiments, the nucleic acid is encapsulated in a lipid nanoparticle. In some embodiments, the method further comprises administering one or more additional HIV-1 immunogens to induce a T cell response.

In certain aspects, the invention provides a composition comprising a nanoparticle and a carrier, wherein the nanoparticle comprises any one of the nucleic acids described herein. In some embodiments, the nanoparticle is a lipid nanoparticle. In some embodiments, the composition is an immunogenic composition.

In certain aspects, the invention provides a method of inducing an immune response in a subject comprising administering an immunogenic composition comprising any one of the recombinant HIV-1 MPER peptides or HIV-1 envelope protein sequences comprising a MPER peptide or any one of the nucleic acids described herein or compositions described herein. In some embodiments, the composition is administered as a prime. In some embodiments, the composition is administered as a boost.

In certain aspects, the invention provides a composition comprising the nucleic acid described herein.

In certain aspects, the invention provides a method of inducing an immune response in a subject comprising administering an immunogenic composition comprising the recombinant HIV-1 MPER peptide or HIV-1 envelope protein sequence comprising a MPER peptide described herein or the nucleic acid described herein.

In some embodiments, the nucleic acid is encapsulated in a lipid nanoparticle.

In certain aspects, the invention provides an immunogenic composition or composition described herein, wherein the composition comprises at least two different HIV-1 MPER peptides or HIV-1 envelope sequences comprising a MPER peptide or nucleic acids encoding a HIV-1 MPER peptide, or HIV-1 envelope comprising a MPER peptide, or a combination thereof.

In certain aspects, the invention provides an immunogenic composition comprising a first immunogen and a second immunogen, wherein the first immunogen is a HIV-1 MPER peptide or HIV-1 envelope protein sequence comprising a MPER peptide comprising all the consecutive amino acids after the signal peptide of SEQ ID NOs: 1, 2 or 16-45, and wherein the second immunogen is a different HIV-1 MPER peptide or HIV-1 envelope protein sequence comprising a MPER peptide comprising all the consecutive amino acids after the signal peptide of SEQ ID NOs: 1, 2 or 16-45. In some embodiments, at least one of the first immunogen and the second immunogen is a recombinant HIV-1 MPER peptide or HIV-1 envelope protein sequence comprising a MPER peptide. In some embodiments, the first immunogen and the second immunogen are both a HIV-1 MPER peptide or HIV-1 envelope protein sequence comprising a MPER peptide. In some embodiments, at least one of the first immunogen and the second immunogen is a nucleic acid. In some embodiments, the nucleic acid is encapsulated in an LNP. In some embodiments, the immunogenic composition further comprises one or more additional immunogens, wherein the one or more additional immunogens is different to the first and second immunogens. In some embodiments, the immunogenic composition comprises a carrier. In some embodiments, the immunogenic composition further comprises an adjuvant.

In certain aspects, the invention provides a method of inducing an immune response in a subject comprising administering the immunogenic composition described herein in an amount sufficient to induce an immune response. In some embodiments, the method further comprises administering an agent which modulates host immune tolerance. In some embodiments, the HIV-1 envelope comprises a MPER peptide comprising all the consecutive amino acids after the signal peptide of SEQ ID NOs: 1 or 2. In some embodiments, the HIV-1 envelope comprises a MPER peptide comprising all the consecutive amino acids after the signal peptide of SEQ ID No: 16-45.

In some embodiments, the HIV-1 envelope is a protomer. In some embodiments, the protomer is comprised in a trimer.

In certain aspects, the invention provides a composition comprising a nanoparticle and a carrier, wherein the nanoparticle comprises any one of the recombinant HIV-1 envelope described herein or the trimer described herein. In some embodiments, the composition is an immunogenic composition. In some embodiments, the nanoparticle is a ferritin self-assembling nanoparticle.

In certain aspects, the invention provides a composition comprising the MPER peptide or HIV-1 envelope protein sequence comprising a MPER peptide described herein.

The invention is described in the following non-limiting examples.

### EXAMPLES

### Example 1

Human B cells arise from committed progenitor cells that proliferate following expression of functional immunoglobulin heavy- (H-) chain polypeptides that associate with surrogate light chains (SLC). In pre-B I cells. H-chain and SLC pairs associate with Igα/Igβ heterodimers to form pre-B cell receptors (pre-BCR) and initiate cell proliferation. When these proliferating cells exit the cell cycle as pre-B II cells, increased RAG1/2 expression drives light- (L-) chain rearrangements and the assembly of mature BCR capable of binding antigen. Most newly generated immature B cells are autoreactive and consequently lost or inactivated at the first tolerance checkpoint; the remainder mature as transitional 1 (T1) and T2 B cells characterized by changes in membrane IgM (mIgM) density, increased mIgD expression, and the loss/diminution of CD10 and CD38. Newly formed T2 B cells are subject to a second round to immune tolerization before entering the mature B cell pools. Mature B cells activated by antigens and TFH characteristically down-regulate mIgD and increase CD38 expression as they enter the germline center (GC) reaction, GC are sites on intense B-cell proliferation, AICDA dependent Ig hypermutation and class-switch recombination, and affinity maturation. See FIG. 1.

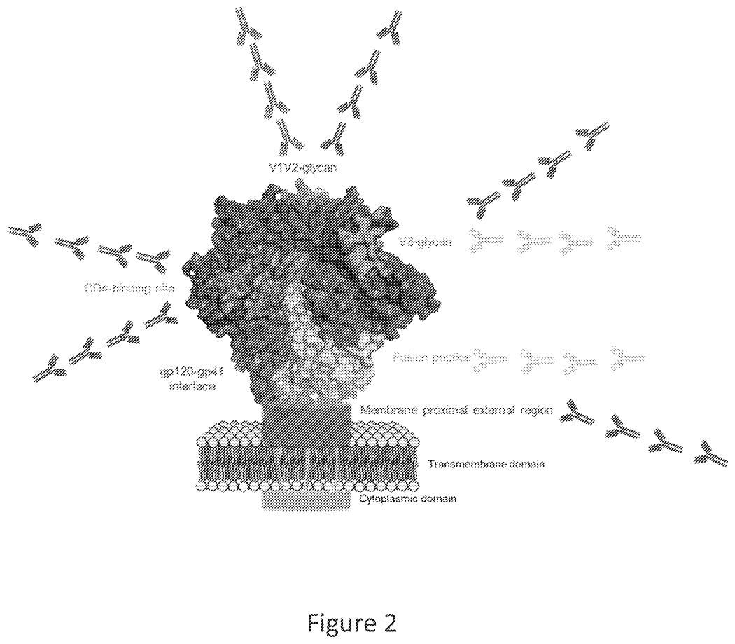

FIG. 2 shows HIV Env trimer with broadly neutralizing targets showing the need for a polyclonal multi-B lineage response to the CD4 binding site bnAb epitope and to at least two other epitopes such as the V1V2 glycan, the V3 glycan, fusion domain or the membrane proximal external region sites.

This example discloses the design and screening of MPER peptide sequences. Four exemplary strategies used to design MPER sequences. FIGS. 3-1 to 3-10. Exemplary MPER peptide sequences or HIV-1 envelope protein sequences comprising a MPER peptide (FIG. 4) are disclosed.

FIGS. 3-1 to 3-10 depict binding data of MPER antibodies binding to cell surface-expressed MPER constructs.

Previously it was observed that HIV-1 Envelope membrane-proximal external region (MPER) has two bnAb epitopes-proximal and distal (closer to the membrane). In HVTN133, germline targeting MPER-peptide liposome induced polyclonal neutralizing responses, ranging from early and late intermediate antibodies to broadly-neutralizing antibodies (bnAbs). In HVTN133, a large bnAb clonal lineage (termed DH1317) demonstrated the full range of neutralization development from precursors to bnAb status.

MPER epitopes include virion lipid. MPER epitopes may be buried in lipids. Mutations may need to be included to expose MPER epitopes from the membrane. Some mutations, such as L669S, have been indicated to have a role in this.

A series of MPER constructs were designed using 4 different strategies. Strategy 1 included either MPER sequence alone, or MPER with envelope (Env) transmembrane domain (TMD), fused to a hydrophobic membrane anchor tag GTH1 or GTH2. Some of them also included a T-helper epitope PADRE (pan-HLA-DR-binding epitope). Since Strategy 1 constructs do not have transmembrane domains they can expressed as protein and/or nucleic acid immunogens. Accordingly, in some embodiments the Strategy 1 constructs provide HIV-1 vaccine immunogens for administration as either protein immunogens or nucleic acid immunogens, e.g., mRNA immunogens. Strategy 2 has MPER sequence fused to TMD truncated at amino acid residue 700; Strategy 3 are MPER sequence fused to TMD and Env cytoplasmic domain (CD); Strategy 4 has full length Env gp160s with various mutations in the transmembrane domain. The designs included both Clade B and Clade C consensus MPER sequences. Since Strategy 2-4 constructs have transmembrane domains they are optimally expressed nucleic acids. Accordingly, in some embodiments the Strategy 2-4 constructs provide HIV-1 vaccine immunogens for administration as nucleic acid immunogens, e.g., mRNA immunogens. A total of 50 MPER designs were screened. See FIG. 3-1.

FIG. 3-2 depicts a workflow of high-throughput screening of MPER antigenicity in 293-F cell transient transfection by flow cytometry. Antigenicity of these MPERs were screened with a panel of antibodies using a 96-well plate-based flow cytometer. This assay allowed screening of a combination of 50 MPERs with 20 antibodies within weeks.

First, the data of 50 MPERs binding to 20 antibodies was plotted in a heatmap. See FIG. 3-3. Data plotted here were averages of log-transformed mean fluorescence intensity (or MFI) in two experiments. Each column is an antibody, labeled at the bottom of the heatmap. The antibodies included MPER bnAbs and bnAb UCAs, and neutralizing antibodies isolated from HVTN133. Each row is an MPER. This color bar on the left annotates the four MPER design strategies above. From this heatmap, it can be seen that the MPERs can roughly be grouped into three categories based on antibody binding: These ones showed no to minimal binding; the ones showed intermediate binding; and the ones showed high binding.

The MPERs with the highest binding are: HV1303005 (B.MPER-TMD), HV1303006 (B.MPER-TMD Y712I), HV1303009 (GTH2-B.MPER-TMD), HV1303010 (GTH2-B.MPER-TMD Y712I), HV1302985 (B.ConMPER-TMD700). See FIG. 3-4. These best binders share some common features: First, they all include the transmembrane domain, but not the cytoplasmic domain; secondly, they all had the Clade B consensus MPER sequence, this is because most MPER antibodies tested require the amino acid Lysine 665 in the LDKW motif (SEQ ID NO: 15), which is a Serine in Clade C consensus sequence; Thirdly, they were able to bind both groups of antibodies that target the proximal and the distal epitopes.

FIG. 3-5 depicts a bar graph showing the binding of HV1303006 (B.MPER-TMD Y712I) to MPER antibodies. Y-axis is MFI in log scale. HV1303006 (B.MPER-TMD Y712I) bound to distal MPER epitope targeting bnAb DH511, but not its UCA. In contrast, it bound to both distal MPER epitope targeting bnAb 2F5, and its UCA, called 2F5 RUA D. Additionally, it bound to neutralizing antibodies isolated from HVTN133.

FIG. 3-6 shows the flow cytometry histograms of a few antibodies. Again, HV1303006 (B.MPER-TMD Y712I) bound to distal MPER epitope bnAb DH511 but not DH511 UCA. It bound to 2F5 and 2F5 RUA D. The binding to 2F5 RUA N was lower. Finally, it showed robust binding to three members of the DH1317 lineage isolated from an HVTN133 donor. Based on these data, the HV1303006 (B.MPER-TMD Y712I) construct may be used as a prime immunogen for the proximal MPER epitope, or as a boost immunogen for HVTN133.

FIGS. 3-7 and 3-8 disclose data of another “high-binding” MPER construct HV1303009 (GTH2-B.MPER-TMD). The results are similar to the previous construct. FIG. 3-7 shows antibody binding to MPER.B-TMD measured by flow cytometry. FIG. 3-8 shows the flow cytometry histogram of MPER.B-TMD binding to selected antibodies.

Epitopes for proximal and distal requires both protein and lipids. FIG. 3-9 discloses another MPER design, HV1302976 (B.ConMPER-TMD-CD Y712I), which falls into the category of “intermediate binding” in the heatmap (FIG. 3-3). The binding of this MPER to MPER antibodies was generally low. However, it bound to both the distal DH511 UCA and the proximal 2F5 RUA.

FIG. 3-10 depicts the HV1302976 histograms. As shown here, the HV1302976 construct bound to DH511 UCA, DH511, 2F5 RUA D, 2F5, and HVTN133 neutralizing antibodies, albeit at lower level.

In summary, 50 MPER constructs were designed and their antigenicity was screened against 20 MPER bnAbs or bnAb UCAs in 293-F cell line. The top candidates HV1303006 (B.MPER-TMD Y712I) and HV1303009 (GTH2-B.MPER-TMD) that maybe used as a prime immunogen for distal MPER epitope or a boost immunogen for HVTN133 were down-selected. HV1302976 that maybe used as a prime immunogen for both distal and proximal MPER epitopes if we can further improve the expression was also down-selected.

Immunization studies in BALB/c or DH511 UCA knock-in mice at 20 μg, 10 μg or 1 μg doses are performed. A series of membrane-bound Env gp160 MPER designs will be screened.

Any one of these immunogens and/or any combination thereof could be tested in any suitable animal study to determine immunogenicity of the envelopes.

What is claimed is:

1. A recombinant HIV-1 MPER peptide comprising all the consecutive amino acids after the signal peptide of SEQ ID NOs: 1, 2, or 16-45 or a HIV-1 envelope protein sequence comprising a MPER peptide comprising all the consecutive amino acids after the signal peptide of SEQ ID NOs: 46-107.

2. The recombinant HIV-1 MPER peptide of claim 1, comprising all the consecutive amino acids after the signal peptide of SEQ ID NO: 28, 29 or 40.

3. A nucleic acid comprising a sequence encoding the recombinant HIV-1 MPER peptide or HIV-1 envelope protein sequence comprising a MPER peptide of claim 1.

4. An immunogenic composition comprising the MPER peptide or HIV-1 envelope protein sequence comprising a MPER peptide of claim 1 and a carrier.

5. An immunogenic composition comprising the nucleic acid of claim 3 and a carrier.

6. The immunogenic composition of claim 4 or 5 further comprising an adjuvant.

7. The nucleic acid of claim 3 or the immunogenic composition of claim 6 wherein the nucleic acid is operably linked to a promoter, and optionally wherein the nucleic acid is inserted in an expression vector.

8. A method of inducing an immune response in a subject comprising administering a composition comprising any suitable form of a nucleic acid(s) of claim 3 or the recombinant HIV-1 MPER peptide or HIV-1 envelope protein sequence comprising a MPER peptide of claim 1 in an amount sufficient to induce an immune response.

9. The method of claim 8, wherein the composition further comprises an adjuvant.

10. The method of claim 8, further comprising administering an agent which modulates host immune tolerance.

11. The method of any of claims 9-10, wherein the nucleic acid is encapsulated in a lipid nanoparticle.

12. The method of any of claims 9-11, further comprising administering one or more additional HIV-1 immunogens to induce a T cell response.

13. A composition comprising a nanoparticle and a carrier, wherein the nanoparticle comprises any one of the nucleic acids of claim 3.

14. The composition of claim 13, wherein the nanoparticle is a lipid nanoparticle.

15. A method of inducing an immune response in a subject comprising administering an immunogenic composition comprising any one of the recombinant HIV-1 MPER peptides or HIV-1 envelope protein sequences comprising a MPER peptide or any one of the nucleic acids of the preceding claims or compositions of the preceding claims.

16. The method of claim 15, wherein the composition is administered as a prime.

17. The method of claim 15, wherein the composition is administered as a boost.

18. A composition comprising the nucleic acid of claim 3.

19. A method of inducing an immune response in a subject comprising administering an immunogenic composition comprising the recombinant HIV-1 MPER peptide or HIV-1 envelope protein sequence comprising a MPER peptide of claim 1 or the nucleic acid of claim 3.

20. The nucleic acid of claim 3, wherein the nucleic acid is encapsulated in a lipid nanoparticle.

21. An immunogenic composition or composition of any of the preceding claims, wherein the composition comprises at least two different HIV-1 MPER peptides or HIV-1 envelope sequences comprising a MPER peptide or nucleic acids encoding a HIV-1 MPER peptide, or HIV-1 envelope comprising a MPER peptide, or a combination thereof.

22. An immunogenic composition comprising a first immunogen and a second immunogen, wherein the first immunogen is a HIV-1 MPER peptide or HIV-1 envelope protein sequence comprising a MPER peptide comprising all the consecutive amino acids after the signal peptide of SEQ ID NOs: 1, 2 or 16-45 or a nucleic acid encoding a HIV-1 MPER peptide or HIV-1 envelope protein sequence comprising a MPER peptide comprising all the consecutive amino acids after the signal peptide of SEQ ID NOs: 1, 2 or 16-45, and wherein the second immunogen is a different HIV-1 MPER peptide or HIV-1 envelope protein sequence comprising a MPER peptide comprising all the consecutive amino acids after the signal peptide of SEQ ID NOs: 1, 2 or 16-45 or a nucleic acid encoding a different HIV-1 MPER peptide or HIV-1 envelope protein sequence comprising a MPER peptide comprising all the consecutive amino acids after the signal peptide of SEQ ID NOs: 1, 2 or 16-45.

23. The immunogenic composition of claim 22, wherein at least one of the first immunogen and the second immunogen is a recombinant HIV-1 MPER peptide or HIV-1 envelope protein sequence comprising a MPER peptide.

24. The immunogenic composition of claim 23, wherein the first immunogen and the second immunogen are both a HIV-1 MPER peptide or HIV-1 envelope protein sequence comprising a MPER peptide.

25. The immunogenic composition of claim 22, wherein at least one of the first immunogen and the second immunogen is a nucleic acid.

26. The immunogenic composition of claim 25, wherein the first immunogen and the second immunogen are both a nucleic acid.

27. The immunogenic composition of claim 21 or 22, wherein the nucleic acid is encapsulated in an LNP.

28. The immunogenic composition according to any one of claims 22 to 27, further comprising one or more additional immunogens, wherein the one or more additional immunogens is different to the first and second immunogens.

29. The immunogenic composition according to any one of claims 22 to 28, wherein the immunogenic composition comprises a carrier.

30. The immunogenic composition according to any one of claims 22 to 29, wherein the immunogenic composition further comprises an adjuvant.

31. A method of inducing an immune response in a subject comprising administering the immunogenic composition according to any one of claims 4-5, or 22-30 in an amount sufficient to induce an immune response.

32. The method of claim 31, further comprising administering an agent which modulates host immune tolerance.

33. The recombinant HIV-1 envelope of claim 1, wherein the HIV-1 envelope comprises a MPER peptide comprising all the consecutive amino acids after the signal peptide of SEQ ID NOs: 1 or 2.

34. The recombinant HIV-1 envelope of claim 1, wherein the HIV-1 envelope comprises a MPER peptide comprising all the consecutive amino acids after the signal peptide of SEQ ID No: 16-45.

35. The recombinant HIV-1 envelope of any one of claims 1, 33, or 34, wherein the HIV-1 envelope is a protomer.

36. The recombinant HIV-1 envelope of claim 35, wherein the protomer is comprised in a trimer.

37. A composition comprising a nanoparticle and a carrier, wherein the nanoparticle comprises any one of the recombinant HIV-1 envelope of claims 1, 33, or 34, or the trimer of claim 36.

38. The composition of claim 37, wherein the nanoparticle is a ferritin self-assembling nanoparticle.

39. A composition comprising the MPER peptide or HIV-1 envelope protein sequence comprising a MPER peptide of claim 1.


SEQUENCE LISTING (107 sequences)

SEQ ID NO: 1 - protein, 28 aa, synthetic construct
NEQELLELDKWASLWNWFNITNWLWYIK

SEQ ID NO: 2 - protein, 28 aa, synthetic construct
NEKDLLALDSWKNLWNWFDITKWLWYIK

SEQ ID NO: 3 - protein, 4 aa, Human immunodeficiency virus
REKR

SEQ ID NO: 4 - protein, 9 aa, Human immunodeficiency virus
RRVVEREKR

SEQ ID NO: 5 - protein, 9 aa, synthetic construct
ERVVEREKE

SEQ ID NO: 6 - protein, 4 aa, synthetic construct
SEKS

SEQ ID NO: 7 - protein, 79 aa, synthetic construct
MRVMGIQRNYPQWWIWSMLGFWMLMICNGMWVTVYYGVPVWKEAKTTLFCASDAKAYEKEVHNVWATHACVPTDPNPQE

SEQ ID NO: 8-11 - skipped (4 entries)

SEQ ID NO: 12 - protein, 24 aa, Homo sapiens
MPMGSLQPLATLYLLGMLVASVLA

SEQ ID NO: 13-14 - skipped (2 entries)

SEQ ID NO: 15 - protein, 4 aa, synthetic construct
LDKW

SEQ ID NO: 16 - protein, 75 aa, synthetic construct
MDSKGSSQKGSRLLLLLVVSNLLLPQGVLANEQELLELDKWASLWNWFNITNWLWYIKYKRWIILGLNKIVRMYS

SEQ ID NO: 17 - protein, 88 aa, synthetic construct
MDSKGSSQKGSRLLLLLVVSNLLLPQGVLANEQELLELDKWASLWNWFNITNWLWYIKYKRWIILGLNKIVRMYSAKFVAAWTLKAAA

SEQ ID NO: 18 - protein, 94 aa, synthetic construct
MDSKGSSQKGSRLLLLLVVSNLLLPQGVLANEQELLELDKWASLWNWFNITNWLWYIKYKRWIILGLNKIVRMYSDYVDRFYKTLRAEQASQEV

SEQ ID NO: 19 - protein, 94 aa, synthetic construct
MDSKGSSQKGSRLLLLLVVSNLLLPQGVLADYVDRFYKTLRAEQASQEVNEQELLELDKWASLWNWFNITNWLWYIKYKRWIILGLNKIVRMYS

SEQ ID NO: 20 - protein, 88 aa, synthetic construct
MDSKGSSQKGSRLLLLLVVSNLLLPQGVLAAKFVAAWTLKAAANEQELLELDKWASLWNWFNITNWLWYIKYKRWIILGLNKIVRMYS

SEQ ID NO: 21 - protein, 77 aa, synthetic construct
MDSKGSSQKGSRLLLLLVVSNLLLPQGVLANEQELLELDKWASLWNWFNITNWLWYIKDYVDRFYKTLRAEQASQEV

SEQ ID NO: 22 - protein, 75 aa, synthetic construct
MDSKGSSQKGSRLLLLLVVSNLLLPQGVLANEKDLLALDSWKNLWNWFDITKWLWYIKYKRWIILGLNKIVRMYS

SEQ ID NO: 23 - protein, 88 aa, synthetic construct
MDSKGSSQKGSRLLLLLVVSNLLLPQGVLANEKDLLALDSWKNLWNWFDITKWLWYIKYKRWIILGLNKIVRMYSAKFVAAWTLKAAA

SEQ ID NO: 24 - protein, 94 aa, synthetic construct
MDSKGSSQKGSRLLLLLVVSNLLLPQGVLANEKDLLALDSWKNLWNWFDITKWLWYIKYKRWIILGLNKIVRMYSDYVDRFYKTLRAEQASQEV

SEQ ID NO: 25 - protein, 94 aa, synthetic construct
MDSKGSSQKGSRLLLLLVVSNLLLPQGVLADYVDRFYKTLRAEQASQEVNEKDLLALDSWKNLWNWFDITKWLWYIKYKRWIILGLNKIVRMYS

SEQ ID NO: 26 - protein, 88 aa, synthetic construct
MDSKGSSQKGSRLLLLLVVSNLLLPQGVLAAKFVAAWTLKAAANEKDLLALDSWKNLWNWFDITKWLWYIKYKRWIILGLNKIVRMYS

SEQ ID NO: 27 - protein, 77 aa, synthetic construct
MDSKGSSQKGSRLLLLLVVSNLLLPQGVLANEKDLLALDSWKNLWNWFDITKWLWYIKDYVDRFYKTLRAEQASQEV

SEQ ID NO: 28 - protein, 91 aa, synthetic construct
MDSKGSSQKGSRLLLLLVVSNLLLPQGVLANEQELLELDKWASLWNWFNITNWLWYIKIFIMIVGGLIGLRIVFAVLSIVNRVRQGYSPLS

SEQ ID NO: 29 - protein, 91 aa, synthetic construct
MDSKGSSQKGSRLLLLLVVSNLLLPQGVLANEQELLELDKWASLWNWFNITNWLWYIKIFIMIVGGLIGLRIVFAVLSIVNRVRQGISPLS

SEQ ID NO: 30 - protein, 104 aa, synthetic construct
MDSKGSSQKGSRLLLLLVVSNLLLPQGVLAAKFVAAWTLKAAANEQELLELDKWASLWNWFNITNWLWYIKIFIMIVGGLIGLRIVFAVLSIVNRVRQGYSPLS

SEQ ID NO: 31 - protein, 104 aa, synthetic construct
MDSKGSSQKGSRLLLLLVVSNLLLPQGVLAAKFVAAWTLKAAANEQELLELDKWASLWNWFNITNWLWYIKIFIMIVGGLIGLRIVFAVLSIVNRVRQGISPLS

SEQ ID NO: 32 - protein, 110 aa, synthetic construct
MDSKGSSQKGSRLLLLLVVSNLLLPQGVLADYVDRFYKTLRAEQASQEVNEQELLELDKWASLWNWFNITNWLWYIKIFIMIVGGLIGLRIVFAVLSIVNRVRQGYSPLS

SEQ ID NO: 33 - protein, 110 aa, synthetic construct
MDSKGSSQKGSRLLLLLVVSNLLLPQGVLADYVDRFYKTLRAEQASQEVNEQELLELDKWASLWNWFNITNWLWYIKIFIMIVGGLIGLRIVFAVLSIVNRVRQGISPLS

SEQ ID NO: 34 - protein, 75 aa, synthetic construct
MDSKGSSQKGSRLLLLLVVSNLLLPQGVLANEQELLELDKWASLWNWFDITNWLWYIKIFIIIVGSLIGLRIVFA

SEQ ID NO: 35 - protein, 75 aa, synthetic construct
MDSKGSSQKGSRLLLLLVVSNLLLPQGVLANEKDLLALDSWKNLWNWFDITKWLWYIKIFIIIVGSLIGLRIVFA

SEQ ID NO: 36 - protein, 75 aa, synthetic construct
MDSKGSSQKGSRLLLLLVVSNLLLPQGVLANEQELLELDKWAKKKNWFDITNWLWYIKIFIIIVGSLIGLRIVFA

SEQ ID NO: 37 - protein, 75 aa, synthetic construct
MDSKGSSQKGSRLLLLLVVSNLLLPQGVLANEKDLLALDSWKKKKNWFDITKWLWYIKIFIIIVGSLIGLRIVFA

SEQ ID NO: 38 - protein, 63 aa, synthetic construct
MDSKGSSQKGSRLLLLLVVSNLLLPQGVLAKKKNWFDITNWLWYIKIFIIIVGSLIGLRIVFA

SEQ ID NO: 39 - protein, 63 aa, synthetic construct
MDSKGSSQKGSRLLLLLVVSNLLLPQGVLAKKKNWFDITKWLWYIKIFIIIVGSLIGLRIVFA

SEQ ID NO: 40 - protein, 163 aa, synthetic construct
MDSKGSSQKGSRLLLLLVVSNLLLPQGVLANEQELLELDKWASLWNWFDITNWLWYIKIFIIIVGSLIGLRIVFAVLSLVNRVRQGISPLSFQTHLPTPRGPDRPEGIEEEGGERDRDRSIRLVNGSLALIWDDLRSLSLFSYHRLRDLLLIVTRIVELLGRR

SEQ ID NO: 41 - protein, 163 aa, synthetic construct
MDSKGSSQKGSRLLLLLVVSNLLLPQGVLANEKDLLALDSWKNLWNWFDITKWLWYIKIFIIIVGSLIGLRIVFAVLSLVNRVRQGISPLSFQTHLPTPRGPDRPEGIEEEGGERDRDRSIRLVNGSLALIWDDLRSLSLFSYHRLRDLLLIVTRIVELLGRR

SEQ ID NO: 42 - protein, 184 aa, synthetic construct
MDSKGSSQKGSRLLLLLVVSNLLLPQGVLANEQELLELDKWASLWNWFDITNWLWYIKIFIIIVGSLIGLRIVFAVLSLVNRVRQGISPLSFQTHLPTPRGPDRPEGIEEEGGERDRDRSIRLVNGSLALIWDDLRSLSLFSYHRLRDLLLIVTRIVELLGRRTVGLYKRWIILGLNKIVRMYS

SEQ ID NO: 43 - protein, 184 aa, synthetic construct
MDSKGSSQKGSRLLLLLVVSNLLLPQGVLANEKDLLALDSWKNLWNWFDITKWLWYIKIFIIIVGSLIGLRIVFAVLSLVNRVRQGISPLSFQTHLPTPRGPDRPEGIEEEGGERDRDRSIRLVNGSLALIWDDLRSLSLFSYHRLRDLLLIVTRIVELLGRRTVGLYKRWIILGLNKIVRMYS

SEQ ID NO: 44 - protein, 180 aa, synthetic construct
MDSKGSSQKGSRLLLLLVVSNLLLPQGVLANEQELLELDKWASLWNWFDITNWLWYIKIFIIIVGSLIGLRIVFAVLSLVNRVRQGISPLSFQTHLPTPRGPDRPEGIEEEGGERDRDRSIRLVNGSLALIWDDLRSLSLFSYHRLRDLLLIVTRIVELLGRRTVGLAKFVAAWTLKAAA

SEQ ID NO: 45 - protein, 180 aa, synthetic construct
MDSKGSSQKGSRLLLLLVVSNLLLPQGVLANEKDLLALDSWKNLWNWFDITKWLWYIKIFIIIVGSLIGLRIVFAVLSLVNRVRQGISPLSFQTHLPTPRGPDRPEGIEEEGGERDRDRSIRLVNGSLALIWDDLRSLSLFSYHRLRDLLLIVTRIVELLGRRTVGLAKFVAAWTLKAAA

SEQ ID NO: 46 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 47 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIRIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 48 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMRVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 49 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIRGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 50 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIVGRLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 51 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIRIVGGLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 52 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMRVGGLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 53 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIRGGLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 54 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIVGRLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 55 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 56 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIRIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 57 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMRVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 58 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIRGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 59 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGRLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 60 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIRIVGGLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 61 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMRVGGLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 62 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIRGGLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 63 - protein, 857 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGCKRRVVEGGGGSGGGGSAAGLGALFLGFLGAAGSTMGAASMTLTVQARQLLGSAGSAGSGSAGSGSAGSGSAGSTVWGIKQLQARVLALERYLKDQQLLGMWGCSGKLICCTNVPWNTSWSNKSETDIWDNMTWMQWEREISNYTETIYKLLEDSQNQQERNEQDLLALDSWNSLWNWFSITKWLWYIKIFIMIVGRLIGLLIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 64 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSLWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 65 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSSWNWFSITKWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 66 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSSWNWFSITKWLWYIKIFIRIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 67 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSSWNWFSITKWLWYIKIFIMRVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 68 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSSWNWFSITKWLWYIKIFIMIRGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 69 - protein, 851 aa, synthetic construct
MDSKGSSQKGSRLLLLLVVSNLLLPQGVLAKGKLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVEIQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSETDIWDNMTWMQWEREISNYTETIYKLLEDSQNQQERNEQDLLALDSWNSSWNWFSITKWLWYIKIFIMIVGRLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGRSSLRGLQRGWEVLKYLGSLVQYWGLELKKSAISLFDTLAIAVAEGTDRIIELIQGFCRAIRNIPTRIRQGFEASLL

SEQ ID NO: 70 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIRIVGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 71 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMRVGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 72 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIRGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 73 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIVGRLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 74 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIRIVGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 75 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMRVGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 76 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIRGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 77 - protein, 839 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIVGRLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 78 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIRIVGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 79 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMRVGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 80 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIRGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 81 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 82 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIRIVGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 83 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMRVGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 84 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIRGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 85 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 86 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIRIVGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 87 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMRVGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 88 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIRGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 89 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 90 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIRIVGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 91 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMRVGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 92 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIRGGLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 93 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 94 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVCTQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKCMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLLIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 95 - protein, 845 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVCTQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKCMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 96 - protein, 845 aa, synthetic construct
MGWSCIILFLVATATGVHAAEQLWVTVYYGVPVWREANTTLFCASDAKAYDTKVRNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVCTQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRWFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKCMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGISPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERAAA

SEQ ID NO: 97 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIRIVGGLVGLRIVCAVLSIVNRVRQGISPLSFQTRLPNPRGPDRPEETEGEGGERDRDRSARLVNGFLAI

SEQ ID NO: 98 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMRVGGLVGLRIVCAVLSIVNRVRQGISPLSFQTRLPNPRGPDRPEETEGEGGERDRDRSARLVNGFLAI

SEQ ID NO: 99 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIRGGLVGLRIVCAVLSIVNRVRQGISPLSFQTRLPNPRGPDRPEETEGEGGERDRDRSARLVNGFLAI

SEQ ID NO: 100 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLRIVCAVLSIVNRVRQGISPLSFQTRLPNPRGPDRPEETEGEGGERDRDRSARLVNGFLAI

SEQ ID NO: 101 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIRIVGGLVGLLIVCAVLSIVNRVRQGISPLSFQTRLPNPRGPDRPEETEGEGGERDRDRSARLVNGFLAI

SEQ ID NO: 102 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMRVGGLVGLLIVCAVLSIVNRVRQGISPLSFQTRLPNPRGPDRPEETEGEGGERDRDRSARLVNGFLAI

SEQ ID NO: 103 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIRGGLVGLLIVCAVLSIVNRVRQGISPLSFQTRLPNPRGPDRPEETEGEGGERDRDRSARLVNGFLAI

SEQ ID NO: 104 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLLIVCAVLSIVNRVRQGISPLSFQTRLPNPRGPDRPEETEGEGGERDRDRSARLVNGFLAI

SEQ ID NO: 105 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVCTQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKCMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIVGRLVGLLIVCAVLSIVNRVRQGISPLSFQTRLPNPRGPDRPEETEGEGGERDRDRSARLVNGFLAI

SEQ ID NO: 106 - protein, 744 aa, synthetic construct
MGWSCIILFLVATATGVHAAEQLWVTVYYGVPVWREANTTLFCASDAKAYDTEVHNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVCTQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKCMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGISPLSFQTRLPNPRGPDRPEETEGEGGERDRDRSARLVNGFLAI

SEQ ID NO: 107 - protein, 744 aa, synthetic construct
MGWSCIILFLVATATGVHAAEQLWVTVYYGVPVWREANTTLFCASDAKAYDTKVRNIWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVCTQVCPKLSFEPIPIHYCAPAGFAILKCNNKTFNGKGPCRNVSTVQCTHGIKPVLSTQLLLNGSLAEEDIIIRSENFTNNGKNIIVQLKEPVKINCTRPGNNTRRSINIGPGRWFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKCMYAPPIRGQIDCSSNITGLILTRDGGSNSSQNETFRPGGGNMKDNWRSELYKYKVVKIEPLGIAPTRCKRRVVQGGGGSGGGGSAVTLGAVFLGFLGAAGSTMGAASMTLTVQARLLLGSAGSAGSGSAGSGSAGSGSAGSTVWGIKQLQARVLAIERYLKDQQLLGIWGCSGKLICCTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGISPLSFQTRLPNPRGPDRPEETEGEGGERDRDRSARLVNGFLAI